(19) United States
(12) Patent Application Publication
(10) Pub. No.: US 2026/0216727 A1
(43) Pub. Date: Jul. 30, 2026

(54) TECHNIQUES FOR MANIPULATING THE CAPILLARY-DRIVEN FLOW

(71) Applicant: GEORGIA TECH RESEARCH CORPORATION, Atlanta, GA (US)

(72) Inventors: Dohwan LEE, Atlanta, GA (US); Ali Faith SARIOGLU, Atlanta, GA (US)

(21) Appl. No.: 19/146,071

(22) PCT Filed: Jan. 5, 2024
(86) PCT No.: PCT/US2024/010481

§ 371 Date: Jul. 7, 2025

Related U.S. Application Data
(60) Provisional application No. 63/437,236, filed on Jan. 5, 2023.

(51) Int. Cl. B01L 3/00; G01N 33/543; G01N 33/569

(52) U.S. Cl. CPC B01L 3/502746; B01L 3/5023; B01L 3/502715; G01N 33/54388; G01N 33/56983; B01L 2200/0647; B01L 2300/0825; B01L 2300/0887; B01L 2300/12; B01L 2300/165; B01L 2400/0406; B01L 2400/086; G01N 2333/11; G01N 2333/165

(57) ABSTRACT

Described herein are microfluidic devices and methods of using thereof. The methods include methods to manipulate capillary-driven flow and methods of detecting an infectious agent.

### CROSS-REFERENCE TO RELATED APPLICATION

This application claims priority to, and the benefit of, U.S. Provisional Application 63/437,236, filed on Jan. 5, 2023, the content of which is hereby incorporated in its entirety.

### REFERENCE TO SEQUENCE LISTING

The Sequence Listing submitted Jan. 5, 2024, as a text filed named “10034_233W01_2024_01_05_Sequence_Listing.xml” created Jan. 5, 2024, and having a file size of 31,573 bytes is hereby incorporated by reference pursuant to 37 C.F.R. § 1.52(e)(5).

### BACKGROUND

The standard platform for point-of-care (POC) testing in resource-limited settings has long been the lateral flow assay (LFA) (Sackmann et al., (2014); Gong et al., (2017); Yetisen et al., (2013); Posthuma-Trumpie et al., (2009)). Lateral flow assays (LFAs) utilize capillary flow of liquids for simple detection of analytes. LFAs offer unique advantages, such as rapid analysis, low-cost, portability, ease-of-use, instrument-free operation, and compatibility with various biological samples (e.g., blood, plasma, serum, urine, sweat, and saliva), all of which make LFAs the predominant diagnostic format for POC applications (Lee et al., (2016); Banerjee et al., (2018); Kong et al., (2017); Verma et al., (2018); Fu et al., (2012); Toley at al., (2013); Lutz et al., (2013)). In fact, LFAs readily satisfy the majority of the ASSURED (Affordable, Sensitive, Specific, User-friendly, Rapid and robust, Equipment-free, and Deliverable to end-users) criteria reported by the World Health Organization in 2004 to establish capabilities of the POC devices (Verma et al., (2018); Fu et al., (2012); Toley at al., (2013); Lutz et al., (2013)). On the other hand, conventional LFAs are less prudent in performing multiplexed assays (e.g., simultaneously screening for multiple analytes within a sample) and are also less sensitive in detecting various analytes of clinical importance (Yetisen et al., (2013); Posthuma-Trumpie, et al., (2009); Lee et al., (2016)).

Microfluidic paper-based analytical devices (pPADs) (Cheng et al., (2010); Martinez et al., (2007); Martinez et al., (2008)) combine the capillary driven flow of LFAs with deterministic liquid routing enabled by microfluidic channels. In pPADs, a liquid-confining channel geometry is created on paper by patterning a hydrophobic material (e.g., photoresist (Martinez et al., (2008)), wax (Lu et al., (2009); Lu et al., (2010)), PDMS (Bruzewicz et al., (2008)), and alkyl ketene dimer (Li et al., (2010))), inkjet printing/etching (Abe et al., (2008); Abe et al., (2010)), plasma or laser treatment (Li, et al., (2008); Li et al., Cellulose (2010); Chitnis et al., (2011)), or stamping (Dornelas et al., (2015)). While channels formed on paper can readily direct multiple liquids to multiple reagents in a pPAD to perform multiplexed assays in parallel, spontaneous capillary flow in paper hinders execution of multi-step bioassays that require timely application of different reagents or buffers in multiple steps, such as immunoassay, enzyme-linked immunosorbent assay (ELISA) and sample purification (Fu et al., (2012); Toley et al., (2013); Lutz et al., (2013); Cheng et al., (2010)).

The lack of an intrinsic mechanism to control capillary-driven liquid flow remains a major bottleneck for LFAs. Recent approaches to control liquid flow in LFAs such as modified paper geometry (Fu et al., (2010); Apilux et al., (2013)), volume-limited source pad (Fu et al., (2012)), tunable-delay shunts (Toley et al., (2013)), sugar deposition (Lutz et al., (2013)), pressurized paper (Shin et al., (2014)), valving function via electromagnet (Li et al., (2013); Fratzl et al., (2018)), fluidic diodes (Chen et al., (2012)), and volume-metered actuation (Kong et al., (2017); Toley et al., (2015)) are highly effective but either require exotic and unscalable fabrication processes hindering their use in practice or large dead volumes limiting their use on manipulating small-volume samples. While useful for spontaneously wicking samples, the capillary flow inherently limits performing complex reactions that require timely application of multiple solutions.

The devices and methods disclosed herein address these and other needs.

### SUMMARY

Provided herein are methods to manipulate capillary-driven flow, the methods including: receiving a liquid sample at a sample inlet of a microfluidic device; flowing the liquid sample along a capillary channel disposed in a substrate of the microfluidic device to a flow delay section of the substrate defined by an area, the area having a delaminating ink forming water-penetrable bonds infused therein; halting the flow of the liquid sample along the substrate at the flow delay section; delaminating the delaminating ink in the area, via the liquid sample interaction with the delaminating ink at a controlled rate to produce flow delay of the liquid sample at the flow delay section; and flowing the liquid sample along another section of the substrate following the flow delay section after a controlled pre-defined time, wherein the liquid sample is subsequently mixed with at least one reagent located in the microfluidic device for analysis (e.g., colorimetry analysis, assay test, chemical amplification) or visualization (e.g., art), and wherein the flow delay section provides a pre-defined time delay for the analysis; wherein the flow delay section includes a diode defined by a taper shaped delaminating ink disposed between two taper shaped non-delaminating ink, wherein the two taper shaped non-delaminating ink are perpendicular to each other, wherein the delaminating ink has a first contact region that expands to a second contact region larger than the first contact region.

Also described are microfluid devices including: a substrate configured to move a fluid by capillary action, the substrate having an inlet area, a test area, and a flow area; adhesive polymetric material adhering to the substrate to define a top and bottom boundaries for capillary flow; a capillary channel disposed in the substrate, the capillary channel defined by a first line of a non-delaminating ink and a second line of the non-delaminating ink infused on the substrate; and a flow delay section defined by an area of the capillary channel, the area having a delaminating ink, wherein the flow delay section includes a diode defined by a taper shaped delaminating ink disposed between two taper shaped non-delaminating ink, wherein the two taper shaped non-delaminating ink are perpendicular to each other, wherein the delaminating ink has a first contact region that expands to a second contact region larger than the first contact region, the delaminating ink being configured to delaminate, via fluid interaction with the delaminating ink, at a controlled rate to produce flow delay of the fluid at the flow delay section.

Also described are methods of detecting an infectious agent including: applying a sample on the device described herein and detecting a signal on the device.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1C shows a tunable flow delay through delamination. (1A) Tunable flow delay on paper by imprinting patterns with two types of water-insoluble inks: non-delaminating (brown) and delaminating ink (green). The channel boundaries were defined with the nondelaminating ink, which created water-impenetrable bonds between the tape and ink-infused paper, permanently tethering each other even when wetted. The timer was drawn with delaminating ink, which created water-penetrable bonds between the tape and ink-infused paper. The swelling of paper in the channel by liquid introduction caused gradual delamination of the timer, producing a flow delay. (1B) A combination of different delaminating inks and laminating tapes with varying contact angles and interfacial adhesion energy modulates the delamination rate, i.e., flow delays. (1C) Time-lapse images taken of a test paper layout when four dye solutions were simultaneously introduced from different inlets. Timers drawn with different delaminating inks (green, red, and black) in each inlet branch differentially delay the capillary flows resulting in a sequential delivery. A control layout on an otherwise identical with no timers resulted in the simultaneous arrival of all dyed solutions to the shared circular channel.

FIGS. 2A-2C shows the mechanism of the delaminating timer. (2A) Time-lapse images of the top and cross-sectional (A-A′) view showing the progress of the dye solution through the timer. The flow was temporarily stopped when it reached the timer, and the swelling of wetted paper derived gradual delamination between the tape and the ink-infused paper. The flow was delayed until the timer was fully delaminated and resumed after the time delay. (2B) Measured surface profiles and height changes at corresponding timepoints. The central area of measurement was swelled by the liquid introduction and eventually delaminated. The channel boundaries at the side of the measured area remained to be clamped with no height change. (2C) Computer simulation to calculate the swelling pressure inducing delamination. Plots showing measured versus simulation-calculated surface profiles and height changes on the x and y-axis at different.

FIGS. 3A-3F shows the characterization of the timer on geometries and delaminating inks. (3A) Measured interfacial adhesion energy Gₜₚ between the laminating tape and the papers infused with three tested delaminating inks. The same laminating tape was used in all measurements (Scotch Heavy Duty Shipping Packaging Tape). (3B) Measured water contact angle on the papers infused with three tested delaminating inks. (3C) Time-lapse images taken of dye solution advancing on four paper lanes, each equipped with a timer of different widths and a different color (green, red, or black). (3D) Measured flow delays (N=10) generated by three different colored timers as a function of their width. The total flow delay increased in quadratic relation with the width of the timer, and the measurements were overlaid with the model. (3E) Time-lapse images taken of dye solution advancing on four paper lanes, each equipped with a different number and color of the cascaded timers. (3F) Measured flow delays (N=10) produced by different colors of cascaded timers as a function of timer number (1 to 8 timers). The total flow delay linearly increased with the number of timers, and the measurements were overlaid with the model.

FIGS. 4A-4D shows a timer characterization of flow delay with different laminating tapes. (4A) Measured interfacial adhesion energy Gₜₚ between the green ink-infused paper and 5 tested laminating tapes. (4B) Images of and (4C) measured water contact angles on the adhesive side of tested tapes before and after corona discharge treatment. The corona treatment rendered all tape surfaces hydrophilic except for Tape #2, which maintained its original contact angle. (4D) Measured flow delays (N=10) produced by 0.5-mm-wide green timer laminated with 5 different tapes before and after corona treatment. As anticipated, corona treatment reduced the flow delays in all tests except for Tape #2. The experimental measurements of delay were overlaid with the model.

FIG. 5. Measured permeability of different colored timers as a function of their width. The local voids formed at the interface between these timers and the laminating tape by delamination were considered as porous media. The average of the measured permeability in each colored timer was used in the derived model.

FIGS. 6A-6C shows a device design and operation. (6A) Photographs showing the device when fully assembled in the 3D-printed case (left) and with its front cover removed (right). A photo of the exposed device shows the back cover holding the internal components (commercial LFA strips of SARS-CoV-2 and influenza A & B and the paint-programmed paper-based flow controller). (6B) A schematic showing an exploded view of the paint-programmed flow controller. Imprinted flow paths and the location of delaminating timer gates within those flow paths are shown for individual layers. Different colored lines in individual layers represent features imprinted with different inks showing varying levels of affinity to polymer tapes when wetted. Locations of cut-outs in each polymer tape are also shown. (6C) A schematic illustrating the process to operate the developed assay.

FIGS. 7A-7C shows programmed capillary flow routing for automated signal amplification. (7A) Time-lapse photos of the assay taken without the cover to show the internal flow controller. The images show the routing of four dye solutions simultaneously introduced from four different inlets. (7B) (Top) Schematics show the in-plane flow paths taken by each amplification reagent as they are delivered to the reaction sequentially, as well as (bottom) the state of the delaminating timer gates at different timepoints in a cross-sectional representation of the programmed flow controller. (7C) A schematic illustrating the chemical amplification process automated by the device for amplifying the colorimetric signal.

FIGS. 8A-8B shows the characterization of the device for SARS-CoV-2 detection. (8A) Close-up photos of the test and control lines of a commercial COVID-19 test strip (top) and the device (bottom) after they were used to process matched control samples of varying SARS-CoV-2 NP concentrations. (8B) A plot showing the normalized intensities measured at the test lines of a commercial LFA (black dotted line) and the assay (red dotted line) as functions of spiked SARS-CoV-2 NP concentration of the control sample. The colorimetric signal level that could not be seen via the naked eye is also marked on the plot.

FIGS. 9A-9D shows characterization of the device for flu detection. (9A) Close-up photos of the influenza test and control lines of a commercial flu test strip (top) and the device (bottom) after they were used to process matched control samples of varying influenza A NP concentrations. (9B) A plot showing the normalized intensities measured at the test lines of a commercial LFA (black dotted line) and the assay (red dotted line) as functions of spiked influenza A NP concentration of the control sample. (9C) Close-up photos of the influenza test and control lines of a commercial flu test strip (top) and the device (bottom) after they were used to process matched control samples of varying influenza B NP concentrations. (9D) A plot showing the normalized intensities measured at the test lines of a commercial LFA (black dotted line) and the assay (red dotted line) as functions of spiked influenza B NP concentration of the control sample. The colorimetric signal level that could not be seen via the naked eye is marked on both plots.

FIGS. 10A-10B shows testing patient samples with the developed technology. (10A) Close-up photos of the test and control lines of a commercial COVID-19 test strip (top) and the device (bottom) after they were used to process matched (left) COVID-19 patient saliva samples (n=3) and (right) control samples collected from healthy donors (n=3). (10B) A plot showing the normalized intensities measured at the test lines of a commercial LFA (grey bars) and the assay (red bars) for processed patient and control samples. The colorimetric signal level that could not be seen via the naked eye is marked on the plot.

FIG. 11 shows photos of individual layers of the designed flow controller showing the imprinted capillary flow paths and timers on each layer. The brown ink defines the flow boundaries on the paper and permanently attaches the laminating tape and the wetted paper. The timer gates (i.e., the roadblocking features of discrete lines drawn normal to the flow direction) were drawn using different inks with different colors. These timers differentially delay the capillary flow.

FIG. 12. Investigation of a potential cross-talk between SARS-CoV-2 and influenza test spots on the device. Close-up photos of the test and control lines for SARS-CoV-2 (left) and influenza viruses (right) of devices that processed control samples spiked with varying concentrations of SARS-CoV-2 NP are shown. No noticeable cross-talk was observed.

FIGS. 13A-13B. Investigation of a potential cross-talk between SARS-CoV-2 and influenza test spots on the device. Close-up photos of the test and control lines for SARS-CoV-2 (left) and influenza viruses (right) of devices that processed control samples spiked with varying concentrations of influenza (13A) A and (13B) BNP are shown. No noticeable cross-talk was observed.

FIGS. 14A-14D shows a fluidic diode using an asymmetric design of a delaminating timer. (14A-14C) A liquid introduced from the left inlet encountered the narrow entry of the diode (i.e., reverse bias) and could not flow through the diode for >60 minutes. Conversely, within the same device, a liquid introduced from the right inlet reached the wider entry of the diode (i.e., forward bias) and could traverse the diode in 3 minutes. (141)) shows a graph with results that the asymmetry in flow delay due to the asymmetric design of the diode could be further enlarged by modifying the design, achieving ~132 min for reverse bias compared to ~3.4 min for forward bias.

FIG. 15 shows a computer simulation of the fluidic diode with an asymmetric design. For the asymmetric design, a trapezoid-shaped diode with dimensions (short base: 0.4 mm, long base: 4 mm, length: 2 mm) was constructed for simulation purposes. The result revealed that surface profile of the diode showed a dome shape upon delamination and a gradual decrease in surface height from long base to short base and from the center to the edge (channel boundary), which demonstrated that the narrower entry of the diode is more resistant to delamination than the wider entry under the same force/pressure.

FIGS. 16A-16B shows a fluidic transistor using back-to-back diodes. The major (large amount of) liquid flow can be controlled by a minor (small amount of) liquid application. The introduction of the small controlling liquid (~3 μl) to the gate activated the fluidic transistor and allowed the major liquid to flow through it (FIG. 16A). As an example application of fluidic transistor, a ‘Yes-or-No-answering’ type of assay (FIG. 16B) was built. If a target analyte is presented in the sample, the assay releases a liquid to ‘YES’ channel representing a positive result, otherwise ‘No’channel for a negative result.

FIG. 17 shows clinical application: one drop operation of LFA with built-in signal amplification. One step-dropping action of the sample can perform both the sample transfer to the LFA strip and sequential delivery of the amplification reagents to the detection area as the sample can be used for rehydration and the liquid carrier of pre-dried reagents.

FIGS. 18A-18D illustrates the concept and underlying principle of the fluidic diode. The flow rate (Q) within the channel patterned on paper using hydrophobic material becomes contingent on the channel width: the flow rate (Q_2) in the narrower channel (w_2) is lower than that (Q_1) in the wider channel (w_1) (FIG. 18A). If the channels were equipped with the delaminating timer, the flow delay by the timer was exponentially increased as the channel width decreased (red line in graph) (FIG. 181B). Based on this fundamental observations, the fluidic diode with asymmetric design of the timer (FIG. 18C) was devised, which functions analogously to an electrical diode. In both test designs featuring two fluidic diodes oriented in opposite directions (circuits 1 and 2), liquids could only pass through the fluidic diode facing the wider entry: red liquid could only pass through a diode 2 (D2), while blue liquid could only pass a diode 1 (D1) (FIG. 18D).

FIGS. 19A-19D shows the implementation of a fluidic transistor and logic gates (or/and gate) using the fluidic diodes. The strategic arrangement of multiple fluidic diodes makes it feasible to implement fluidic transistors and logic gates on paper that behave similarly to electrical transistors and logic gates. For the configuration of the fluidic transistor, two fluidic diodes were positioned in opposite directions within the same channel, creating a base channel between them (FIGS. 19A and 19B). For the configuration of the fluidic OR gate, two fluidic transistors were positioned in parallel, sharing a common source and output (FIG. 19C). Similarly, for the configuration of the fluidic AND gate, two fluidic transistors were connected in serial (FIG. 19D).

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

General Definitions As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a,” “an,” and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient.

The term “patient” refers to a subject under the treatment of a clinician, e.g., a physician. Administration of the therapeutic agents can be carried out at dosages and for periods of time effective for the treatment of a subject. In some embodiments, the subject is a human.

Reference will now be made in detail to specific aspects of the disclosed materials, compounds, compositions, articles, and methods, examples of which are illustrated in the accompanying Examples and Figures.

### Devices

Described herein are microfluid devices including: a substrate configured to move a fluid by capillary action, the substrate having an inlet area, a test area, and a flow area; adhesive polymetric material adhering to the substrate to define a top and bottom boundaries for capillary flow; a capillary channel disposed in the substrate, the capillary channel defined by a first line of a non-delaminating ink and a second line of the non-delaminating ink infused on the substrate; and a flow delay section defined by an area of the capillary channel, the area having a delaminating ink, wherein the flow delay section includes a diode defined by a taper shaped delaminating ink disposed between two taper shaped non-delaminating ink, wherein the two taper shaped non-delaminating ink are perpendicular to each other, wherein the delaminating ink has a first contact region that expands to a second contact region larger than the first contact region, the delaminating ink being configured to delaminate, via fluid interaction with the delaminating ink, at a controlled rate to produce flow delay of the fluid at the flow delay section.

In further embodiments, the case comprises at least one sample inlet. Sample inlet refers to the inlet of the device described herein in which the sample is deposited when using the device.

In certain embodiments, the case comprises at least one auxiliary inlet. Auxiliary inlet refers to the inlet of the device described herein in which the reagents used when operating the device are deposited.

In specific embodiments, the case comprises at least one detection window. Detection window refers to an aperture in the case through which the test line and control line can be observed when using the device.

In some embodiments, the device further comprises at least one signal amplification reagent, wherein the signal amplification reagent is deposited in the auxiliary inlet. As used herein, signal amplification reagents can include, but are not limited to, enhancers, activators, initiators, or buffers. These agents can include, but are not limited to, Proteinase K, deionized water, tris-EDTA buffer, or gold enhancement reagents, such as gold-based auto metallography reagents.

In further embodiments, the device further comprises at least one control line. The control line refers to the line that indicates that the sample has flowed through the test strip.

In some embodiments, the device further includes more than two capillary channels. In some embodiments, the device further includes three capillary channels. In some embodiments, a first capillary channel is a sample channel in fluid communication with an inlet area defined by a first line of a non-delaminating ink and a second line of the non-delaminating ink disposed on the substrate. In some embodiments, a second capillary channel is a first reagent channel in fluid communication with the inlet area defined by a third line of a non-delaminating ink and a fourth line of the non-delaminating ink disposed on the substrate. In some embodiments, a third capillary channel is a second reagent channel in fluid communication with the inlet area defined by a fifth line of a non-delaminating ink and a sixth line of the non-delaminating ink disposed on the substrate.

In some embodiments, the device further includes a first fluidic diode disposed on the sample channel, a second fluidic diode disposed on the first reagent channel, and a third fluidic diode disposed on the second reagent channel.

In some embodiments, the device further includes an ink-based transistor formed by having at least two diodes opposite to each other, wherein the flow is controlled by a liquid application at a gate between the two fluidic diodes.

In some embodiments, the ink-based transistor includes an input, a gate, and an output, wherein a fluid flow to the gate allows a fluid sample to flow from the input to the output.

In some embodiments, the device further includes a plurality of ink-based transistors arranged in a serial arrangement or parallel arrangement.

In some embodiments, the first reagent channel includes a first reagent including a dried amplification reagent A. In some embodiments, the second reagent channel includes a second reagent including a dried amplification reagent B. In some embodiments, the first and second reagents are sequentially delivered to the sample channel upon contact with a liquid sample in the inlet. In some embodiments, the first and second reagents amplify the colorimetric signal.

In some embodiments the amplification reagent A or reagent B can include gold nanoparticles (AuNPs).

In some embodiments, the device further includes laminating tape adhering to the substrate to define a top and bottom boundaries for capillary flow. As used herein, a laminating tape refers to a material that is capable of holding materials together in a functional manner. In some embodiments, laminating tape can include tape. In further embodiments, the laminating tape can be chemically active such that it can selectively cause the ink to delaminate faster, slower, or not at all based on the chemical content of the sample.

As used herein, substrate can include porous materials such as paper, cellulose fiber filters, or woven meshes. In some embodiments, the substrate is a cellulose substrate. In some embodiments, the device can be multilayered such that the laminating tape and substrates can be alternatively stacked on top of each other. In some embodiments, the substrate is permeable to a sample.

As used herein, non-delaminating ink refers to ink that is comparatively more hydrophobic than delaminating ink and remains tethered to an adhesive material for a longer period of time than the delaminating ink. In certain embodiments, non-delaminating ink can be used to create channel boundaries to guide the flow of the liquid in the device.

In some embodiments, it is required that the non-delaminating ink is more hydrophobic than the delaminating ink. In further embodiments, the non-delaminating ink can eventually become untethered from the laminating tape when the sample has fully flowed through the device.

In certain embodiments, the non-delaminating ink includes dye. In specific embodiments, the non-delaminating ink does not include dye.

As used herein, channel refers to a feature on or in an article or the substrate that at least partially directs the flow of a fluid.

As used herein, delaminating ink refers to ink that is comparatively less hydrophobic than non-delaminating ink and separates from an adhesive material more quickly than the non-delaminating ink. Herein, delaminating ink can be used to temporarily hold the flow of liquid in the device. Delaminating ink is used in some embodiments of the invention as a line that extends between the boundaries of the channels and herein can also be referred to as a timer. In certain embodiments, the delaminating ink includes dye. In specific embodiments, the delaminating ink does not include dye.

In some embodiments, the sample includes an environmental sample. In some embodiments, the sample includes a biological sample.

In some embodiments, the device comprises a lateral flow assay. A lateral flow assay (LFA) is a paper-based platform for the detection and quantification of analytes in a mixture, where the sample is placed on a test device, and the results are then displayed. LFAs can be used for the qualitative and/or quantitative detection of specific antigens and antibodies, as well as products of gene amplification. Biological samples that can be tested using LFAs include, but are not limited to, urine, saliva, sweat, serum, plasma, whole blood, or any combination thereof.

In further embodiments, the non-delaminating ink comprises a hydrophobic resin. As used herein, the hydrophobic resin is a resin with a low affinity to water.

In further embodiments, the laminating tape is treated with corona discharge. Corona discharge is a moderately low-power electric discharge that can occur at or near atmospheric pressure. The corona can be produced by strong electric fields associated with small diameter wires, needles, or sharp edges on an electrode. The free electron density in corona discharges is approximately 108 electrons/cm³. Corona discharge occurs when an electrode at a high electric potential ionizes the gas surrounding it. The gas discharges the potential. If a plastic film is passed between the high-potential electrode and a grounded electrode, some of the ionized gas particles will undergo chemical reactions with the plastic surface, introducing reactive groups to the surface and increasing surface roughness. Functional groups such as carbonyls, hydroxyls, hydroperoxides, aldehydes, ethers, esters, carboxylic acids, and unsaturated bonds can be produced during this process. Corona discharge treatment has industrial applications, which include electrophotography, printers, textile processing, and in-powder coating. Corona discharge can operate in atmospheric pressure, so air can be used as a reagent gas. Air plasma treatment can tailor the physical and chemical properties of the surface of materials, resulting in enhanced hydrophilicity of the material by incorporating a variety of polar functional groups, such as oxygen and nitrogen-containing groups.

In certain embodiments, the device further comprises chemicals to modulate adhesion and/or hydrophobicity. Chemicals that modulate adhesion can include, but are not limited to, adhesive polymers, such as acrylic, polyethylene, polystyrene, or any combination thereof. Chemicals that modulate hydrophobicity can include, but are not limited to, hydrophilic or hydrophobic chemicals, such as polyvinyl alcohol, trichloro(octyl(silane, or any combination thereof. The use of chemicals that modulate adhesion and/or hydrophobicity can help to control the required energy for delamination.

In specific embodiments, the substrate is treated with biomolecules. As used herein, biomolecules can modulate the affinity between the adhesive materials and the substrates. In some embodiments, the biomolecules comprise a set of biomolecules. In further embodiments, sets of biomolecules can include an antigen and an antibody, biotin and streptavidin, an enzyme and a substrate, or hybridization of DNA. In modulating the affinity between the laminating tape and the substrates, the biomolecules manipulate the levels of delamination.

In some embodiments, a contact angle between the delaminating ink and the substrate is from 0° to 180°. Contact angle is a measure of the ability of a liquid to wet the surface of a solid. The contact angle is an angle formed by a liquid at the three-phase boundary where a liquid, gas, and solid intersect. As the contact angle decreases, surface energy increases, and surface tension decreases. In some embodiments, the contact angle is from 0° to 60°, 600 to 120°, or 1200 to 180°. In further embodiments, the contact angle is from 0° to 20°, 200 to 40°, 400 to 60°, 600 to 80°, 800 to 100°, 1000 to 120°, 1200 to 140°, 1400 to 160°, or 1600 to 180°. In specific embodiments, the contact angle is from 0° to 30°, 0° to 60°, 0° to 90°, 0° to 120°, 0° to 150°, or 0° to 180°. In some embodiments, the contact angle is from 0° to 15°, 150 to 30°, 300 to 45°, 450 to 60°, 600 to 75°, 750 to 90°, 90° to 105°, 105° to 120°, 120° to 135°, 135° to 150°, 150° to 165°, or 165° to 180°. In certain embodiments, the contact angle is from 900 or less, 800 or less, 700 or less, 600 or less, 500 or less, 400 or less, 300 or less, 200 or less, or 100 or less.

### Methods

### Method to Manipulate Capillary-Driven Flow

Described herein are methods to manipulate capillary-driven flow. In some embodiments, the method can include: receiving a liquid sample at a sample inlet of a microfluidic device; flowing the liquid sample along a capillary channel disposed in a substrate of the microfluidic device to a flow delay section of the substrate defined by an area, the area having a delaminating ink forming water-penetrable bonds infused therein; halting the flow of the liquid sample along the substrate at the flow delay section; delaminating the delaminating ink in the area, via the liquid sample interaction with the delaminating ink at a controlled rate to produce flow delay of the liquid sample at the flow delay section; and flowing the liquid sample along another section of the substrate following the flow delay section after a controlled pre-defined time, wherein the liquid sample is subsequently mixed with at least one reagent located in the microfluidic device for analysis (e.g., colorimetry analysis, assay test, chemical amplification) or visualization (e.g., art), and wherein the flow delay section provides a pre-defined time delay for the analysis.

In some embodiments, the flow delay section can include a diode defined by a taper-shaped delaminating ink disposed between two taper-shaped non-delaminating ink. In some embodiments, the two taper-shaped non-delaminating ink are perpendicular to each other.

In some embodiments, the delaminating ink has a first contact region that expands to a second contact region larger than the first contact region.

In some embodiments, the device can further include a plurality of ink-based transistors arranged in a serial arrangement or parallel arrangement.

In some embodiments, the ink-based transistor can be formed by having at least two diodes opposite to each other. In some embodiments, the flow can be controlled by liquid application at a gate between the two fluidic diodes.

In some embodiments, the ink-based transistor can include an input, a gate, and an output. In some embodiments, a fluid flow to the gate allows the fluid sample to flow from the input to the output.

In some embodiments, the reagent amplifies a colorimetric signal. In some embodiments, the liquid sample is subsequently mixed with at least one reagent located in the microfluidic device for visualization (e.g., art).

In some embodiments, the method can further include: guiding the flow of the liquid sample along the capillary channel and/or flow delay section with a non-delaminating ink infused into the substrate to form boundaries of the capillary channel and/or flow delay section.

In some embodiments, the capillary channel includes a first line of a non-delaminating ink and a second line of the non-delaminating ink infused on the substate.

In some embodiments, the flow delay section includes a line of delaminating ink extending between the first line and the second line of non-delaminating ink. In some embodiments, the flow delay section includes more than two lines of the delaminating ink extending between the first line and the second line of non-delaminating ink. In some embodiments, the non-delaminating ink includes a hydrophobic resin.

In some embodiments, the method further including: flowing the liquid sample along a second capillary channel; and testing the liquid sample of the second capillary channel in the analysis or visualization prior to the liquid sample of the first capillary channel due to the pre-defined time delay provided to the first capillary channel by the flow delay section.

### Method of Detecting an Infectious Agent

Described are also methods of detecting an infectious agent including: applying a sample on the device described herein and detecting a signal on the device.

In some embodiments, the infectious agent is a virus. In some embodiments, one or more infectious agents are detected. In some embodiments, one or more viruses are detected. In some embodiments, the virus is SARS-Cov-2 virus, an influenza A virus, an influenza B virus, Zika virus, HIV, or hepatitis B virus. In some embodiments, the virus is SARS-Cov-2 virus, an influenza A virus, or an influenza B virus.

In some embodiments, a method is used wherein the sample includes urine, blood, plasma, serum, sweat, saliva, or any combination thereof. In some embodiments, a method is used wherein the sample includes saliva.

In further embodiments, a method of detecting an infectious agent includes collecting a sample (e.g., nasal swab, saliva swab, blood sample, etc.), depositing a sample in the device (e.g., in a sample inlet), depositing reagents (e.g., proteinase K, deionized water, RT-LAMP buffer) in the device (e.g., in separate auxiliary inlets), depositing three primer mixtures in the device, wherein the primer mixtures target SARS-CoV-2, influenza A, and influenza B, heating the device to a desired temperature using the internal heat module, and detecting the presence of a signal (e.g., colorimetric, fluorescent) for a target (e.g., SARS-CoV-2, influenza A, and/or influenza B). In certain embodiments, the samples can be deposited in the device in the form of DNA extraction spots. In specific embodiments, images of the signals can be captured using the ImageJ program. In some embodiments, the sample is subjected to the reagents in the following order: proteinase K, deionized water, and RT-LAMP buffer, due to the inclusion of timers in the device.

In some embodiments, the first primer mixture targets the SARS-CoV-2 virus. Primer mixture refers to a mixture comprising a nucleic acid molecule including DNA, RNA, or analogs thereof, suitable for DNA-related techniques including, but not limited to, hybridization, strand extension, amplification, or sequencing. In some embodiments, primers can be DNA. In further embodiments, primers can be single-stranded DNA. In certain embodiments, primers can be from 18 to 25 nucleotides long.

Severe acute respiratory syndrome coronavirus 2 (SARS-CoV-2) is a type of huma coronavirus. Representative examples of human coronavirus can also include, but are not limited to, human coronavirus 229E (HCoV-229E), human coronavirus OC43 (HCoV-OC43), human coronavirus HKU1 (HCoV-HKU1), Human coronavirus NL63 (HCoV-NL63), severe acute respiratory syndrome coronavirus (SARS-CoV), and Middle East respiratory syndrome-related coronavirus (MERS-CoV).

In some embodiments, the coronavirus infection can be caused by an avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV.

As used herein, “COVID-19” refers to the infectious disease caused by SARS-CoV-2 and characterized by, for example, fever, cough, respiratory symptoms, rhinorrhea, sore throat, malaise, headache, chills, repeated shaking with chills, diarrhea, new loss of smell or taste, muscle pain, or a combination thereof.

In some embodiments, the subject with a coronavirus exhibits one or more symptoms associated with mild COVID-19, moderate COVID-19, mild-to-moderate COVID-19, severe COVID-19 (e.g., critical COVID-19), or exhibits no symptoms associated with COVID-19 (asymptomatic). It should be understood that in reference to the treatment of patients with different COVID-19 disease severity, “asymptomatic” infection refers to patients diagnosed with COVID-19 by a standardized RT-PCR assay that does not present with fever, cough, respiratory symptoms, rhinorrhea, sore throat, malaise, headache, or muscle pain.

In some embodiments, the subject with a coronavirus exhibits one or more symptoms selected from dry cough, shortness of breath, and fever. In other embodiments, the subject exhibits no symptoms associated with COVID-19 but has been exposed to another subject known or suspected of having COVID-19.

In further embodiments, the first primer mixture comprises a polynucleotide having an amino acid identified by any one of SEQ ID NOS: 1-12. In further embodiments, the first primer mixture comprises a polynucleotide having an amino acid identified by any one of SEQ ID NOS: 1-6. In further embodiments, the first primer mixture comprises a polynucleotide having an amino acid identified by any one of SEQ ID NOS: 7-12.

In certain embodiments, the second primer mixture targets influenza A virus. Influenza A virus is a negative-sense, single-stranded, segmented RNA virus. The subtypes are labeled according to an H number (for the type of hemagglutinin) and an N number (for the type of neuraminidase). There are 18 different known H antigens and 11 different known N antigens. Influenza A can cause influenza in birds and some mammals. Strains of all subtypes of influenza A virus have been isolated from wild birds. Some isolates of influenza A virus cause severe disease in domestic poultry, and, rarely, in humans. The virus can be transmitted from wild aquatic birds to domestic poultry, which can cause an outbreak or give rise to human influenza pandemics. Influenza A includes avian influenza, which further includes, but is not limited to, subtypes such as H6N1, H6N2, H7N6, H7N7, H7N9, H9N6, H9N7, H9N8, H9N9, H10N3, H10N4, H10N5, H10N6, H10N7, and H10N8.

In specific embodiments, the second primer mixture comprises a polynucleotide having an amino acid identified by any one of SEQ ID NOS: 13-18.

In some embodiments, the third primer mixture targets the influenza B virus. Influenza B is a type of influenza that can only pass from human to human. Influenza B can be highly contagious and can have dangerous effects in a patient's health in more severe cases. Symptoms of influenza B can include, but are not limited to, fever, chills, sore throat, coughing, runny nose and sneezing, fatigue, and muscle aches and body aches. The influenza B genome is

In further embodiments, the third primer mixture comprises a polynucleotide having an amino acid identified by any one of SEQ ID NOS: 19-24.

Primer sequences are listed in Table 1 and the accompanying sequence listing.

TABLE 1
Primer sequences targeting SARS-CoV-2, influenza A and B viruses

| Virus | Gene | Primer | Sequence |
|---|---|---|---|
| SARS-CoV-2 | Nucleocapsid | F3 | ACCAGGAACTAATCAGACAAG (SEQ ID NO: 1) |
| | 2 (N2) | B3 | GACTTGATCTTTGAAATTTGGATCT (SEQ ID NO: 2) |
| | | FIP | TTCCGAAGAACGCTGAAGCGGAACTGATTACAAACATTG |
| | | | GCC (SEQ ID NO: 3) |
| | | BIP | CGCATTGGCATGGAAGTCACAATTTGATGGCACCTGTGT |
| | | | A (SEQ ID NO: 4) |
| | | LF | GGGGGCAAATTGTGCAATTTG (SEQ ID NO: 5) |
| | | LB | CTTCGGGAACGTGGTTGACC (SEQ ID NO: 6) |
| | Envelope | F3 | TGAGTACGAACTTATGTACTCAT (SEQ ID NO: 7) |
| | 1 (E1) | B3 | TTCAGATTTTTAACACGAGAGT (SEQ ID NO: 8) |
| | | | ACCACGAAAGCAAGAAAAAGAAGTTCGTTTCGGAAGAG |
| | | FIP | ACAG (SEQ ID NO: 9) |
| | | BIP | TTGCTAGTTACACTAGCCATCCTTAGGTTTTACAAGACTC |
| | | | ACGT (SEQ ID NO: 10) |
| | | LF | CGCTATTAACTATTAACG (SEQ ID NO: 11) |
| | | LB | GCGCTTCGATTGTGTGCGT (SEQ ID NO: 12) |
| Influenza | Hemagglutinin | F3 | GCTAAGAGAGCAATTGAGC (SEQ ID NO: 13) |
| A | (HA) | B3 | ATGTAGGATTTGCTGAGCT (SEQ ID NO: 14) |
| | | FIP | GGTTT (SEQ ID NO: 15) |
| | | | CGAGTCATGATTGGGCCATGACAGTGTCATCATTTGAAA |
| | | | AAGGTGTAACGGCAGCATGTCCGAATTTCCTTTTTTAAC |
| | | BIP | TAGCCAT (SEQ ID NO: 16) |
| | | LF | ACTTGTCTTGGGGAATATCTC (SEQ ID NO: 17) |
| | | LB | ATGCTGGAGCAAAAAGCT (SEQ ID NO: 18) |
| Influenza | Non-structural | F3 | AGGGACATGAACAACAAAGA (SEQ ID NO: 19) |
| B | protein | B3 | CAAGTTTAGCAACAAGCCT (SEQ ID NO: 20) |
| | 1 (NS1) | | TCAGGGACAATACATTACGCATATCGATAAAGGAGGAA |
| | | FIP | GTAAACACTCA (SEQ ID NO: 21) |
| | | BIP | TAAACGGAACATTCCTCAAACACCACTCTGGTCATAGGC |
| | | | ATTC (SEQ ID NO: 22) |
| | | LF | TCAAACGGAACTTCCCTTCTTTC (SEQ ID NO: 23) |
| | | LB | GGATACAAGTCCTTATCAACTCTGC (SEQ ID NO: 24) |

In certain embodiments, the device further comprises Proteinase K and DI water disposed on the first substrate side.

In specific embodiments, the device further comprises RT-LAMP buffer disposed on the third substrate side. RT-LAMP (reverse transcription loop-mediated isothermal amplification) buffer is a buffer chemical used for an RT-LAMP reaction. RT-LAMP is a method of amplifying and identifying the transcripts of a targeted pathogen. More specifically, RT-LAMP can be used for viral RNA detection.

In some embodiments, the device further comprises an internal heating element coupled to the second adhesive material opposite the fourth substrate side. In further embodiments, the internal heating element comprises an exothermic reaction. In certain embodiments, the exothermic reaction comprises an oxidation reaction. In specific embodiments, the oxidation reaction comprises calcium oxide and water.

As used herein, the internal heating element can include an internal heating module with an exothermic reaction that when triggered, produces the appropriate heat, and can initiate the RT-LAMP reaction. An exothermic reaction is a reaction in which energy is released into the surroundings in the form of light or heat. Exothermic reactions can include, but are not limited to, combustion, polymerization, neutralization, respiration, and oxidation. An oxidation reaction is a type of chemical reaction that involves a transfer of electrons between two species and as such, a change in the oxidation number of a molecule, atom, or ion by gaining or losing an electron. Oxidation reactions include combination reactions, which entail the combining of elements to form a chemical compound. Oxidation reactions can include the reaction between calcium oxide and water to form calcium hydroxide, as demonstrated below.

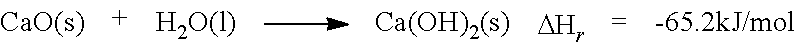

In one embodiment, calcium oxide powder can be made to react with water continuously carried from a neighboring reservoir via a cellulose paper strip (FIG. 20A). In further embodiments, temperature regulation of the reaction can be accomplished with a candelilla wax that surrounds the reaction chamber in which the reaction takes place, wherein the wax melts at from approximately 68° C. to 72° C. In some embodiments, the wax melts at from 68° C. to 69° C., 69° C. to 70° C., 70° C. to 71° C., or 71° C. to 72° C.

The internal heating element can also include a different exothermic reaction, wherein the exothermic reaction provides the required heat for the RT-LAMP reaction. In further embodiments, the internal heating element can include an electric heating element. In some embodiments, the internal heating element includes a regulator, such as candelilla wax.

A number of embodiments of the disclosure have been described. Nevertheless, it will be understood that various modifications may be made without departing from the spirit and scope of the invention. Accordingly, other embodiments are within the scope of the following claims.

The compositions and methods of the appended claims are not limited in scope by the specific compositions and methods described herein, which are intended as illustrations of a few aspects of the claims, and any compositions and methods that are functionally equivalent are intended to fall within the scope of the claims. Various modifications of the compositions and methods, in addition to those shown and described herein, are intended to fall within the scope of the appended claims. Further, while only certain representative compositions and method steps disclosed herein are specifically described, other combinations of the compositions and method steps are also intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

By way of non-limiting illustration, examples of certain embodiments of the present disclosure are given below.

### Examples

Here, a simple, scalable method to manipulate the capillary-driven flow using a tunable delaminating timer imprinted on the paper was introduced. By tuning the timer geometry and the rate of delamination using different sets of inks and tapes with varying material properties, tunable flow delay on paper ranging from tens of seconds to minutes and even hours can be created.

A multiplexed lateral flow assay (LFA) for the detection of SARS-CoV-2, influenza A, and B viruses in human saliva with a built-in chemical amplification of colorimetric signal for enhanced sensitivity is also described. Using this assay, three viruses can be detected with ~25×higher sensitivity than commercial LFAs and showed that the device can detect SARS-CoV-2-positive patient saliva samples missed by commercial LFAs.

An asymmetric design of a tunable delaminating timer can be used to create a fluidic diode and transistor. By tuning the channel width asymmetrically, the transistor can drastically increase/decrease the flow delay depending on the direction flow of the fluid. In addition, coupling two fluidic diodes in a back-to-back manner enables the building of the fluidic transistor: the major (large) liquid flow can be controlled by a minor (small) liquid application.

Prior references of paper-based diagnostic devices lack an intrinsic mechanism to manipulate the capillary-driven flow, hindering the execution of multistep bioassays that require the timely application of different reagents or buffers in multiple steps, such as immunoassay, enzyme-linked immunosorbent assay (ELISA), and sample purification. Although some approaches to control capillary flow were suggested, they still require either exotic and unscalable fabrication processes hindering their use in practice or large dead volumes limiting their use in processing small-volume samples.

The advantage over prior references is that the paper can be programed by simply drawing the timers at strategic nodes to coordinate different capillary flows, sequentially introduce different reagents into a reaction, leaving optimal incubation times in between, and autonomously perform complex assays that could otherwise not be possible with conventional LFAs. As one of the applications of the techniques, SARSCoV-2, influenza A and B viruses can be detected with ~25× higher sensitivity than commercial LFAs. The device described can detect SARS-CoV-2-positive patient saliva samples missed by commercial LFAs.

The exemplary technique can make it possible to develop lateral flow assays that can rival ELISA- or PCR-based assays for sensitive and specific detection of pathogenic targets such as Zika virus, HIV, hepatitis B virus, or diseases like malaria. As a demonstration, a multiplexed lateral flow assay was developed for the detection of SARS-CoV-2 and influenza A and B viruses with ~25× higher sensitivity than commercial LFAs. This technique has the potential to transform a variety of biological assays and tests exclusively performed at clinical laboratories into single-use, disposable dipstick tests to be used at the point of care or at home.

### Example 1: Tunable Capillary Flow on Paper Through Imprinted Timers

While cellulose paper has long been a promising material for sensor applications in biomedical fields, its spontaneous capillary action limits performing assays that require the timely delivery of multiple reagents. Here, a simple, scalable method is provided to control liquid flow on paper using a tunable delaminating timer imprinted on laminated paper. The timer imprinted at strategic nodes using water-insoluble inks produces the flow delay for the desired period through the gradual delamination between ink-infused paper and laminating tape. By tuning the timer geometry and the rate of delamination using different sets of inks and tapes with varying material properties, tunable flow delay on paper can be created ranging from tens of seconds to minutes and even hours. Considering how prominent paper is as a substrate material in analytical and biomedical applications, this work has the potential traditionally labor-intensive assays available in simple paper formats.

### INTRODUCTION

Paper, a thin sheet of cellulose fiber, has long been a material widely used for sensor applications in biomedical fields. Besides its characteristics of disposability, low cost, and biocompatibility, the paper has several advantages that make it a great potential material to be used in sensing devices [1-5]. In particular, spontaneous liquid wicking by capillary action makes the paper a promising material for point-of-care (POC) testing [1-5], mainly operated by transporting biological samples from its source to the region of testing without relying on any external fluidic accessories such as tubing, valve, and pump. Since the concept of lateral flow assays (LFAs) was introduced in 1949, LFAs become the gold standard platforms in POC testing [2-4] indeed, LFAs were the most common POC devices for diagnosis of pandemic viruses [6, 7]. However, conventional LFAs tend not to satisfy minimal criteria in the diagnosis of clinically important diseases due to low sensitivity with a high risk of false negative results and the lack of capability in multiplexed assays [8-12].

Microfluidic paper-based analytical devices (PADs) make LFAs available in multiplexed assays by the deterministic flow routing to multiple directions by microfluidic channels [8-10]. The liquid confining (micron-sized) channels are typically created on paper by patterning hydrophobic materials such as photoresist, polydimethylsiloxane, or wax [14, 15]. The manipulation of capillary flow in designated directions provides advantages that (1) sample/reagent can be concurrently delivered to multiple regions with desired concentrations and (2) sample dead volume can be minimized, especially useful in volume-limited samples, e.g., tears, saliva, sweat, urine, and blood from neonates, all of which enable multiplexed and repetitive assays on a single device [8, 9].

While PADs are effective in performing multiplexed assays in parallel, spontaneous capillary flow with no flow control mechanism (i.e., stop and resume the flows at desired timepoints) limits the implementation of time-sensitive and multistep bioassays on paper [5, 16-21]. Previous approaches to control capillary flow on the paper modulating paper's effective pore size/geometry [18, 19], liquid viscosity [20], surface chemical property [21], or employing mechanical actuators such as an electromagnet [22, 23] or shape-memory polymer [24], require either exotic and unscalable fabrication/treatment hindering their use in practice and on processing small-volume samples.

A study was conducted to introduce a new scalable method to achieve the tunable capillary flow delay on paper for the desired periods through the delamination of wetted paper from laminating tape. Timers producing the controlled delay were drawn on laminated paper using water-insoluble ink, and the ink-patterned paper was delaminated from the tape due to swelling of wetted paper. This process gradually forms local voids at the interface between the timer and the tape as the path for the liquid flow, producing the controlled amount of delays. To understand the factors affecting the flow delay, the study derived a mathematical model that implies that the delay is tunable either by modifying timer geometry or material properties of the ink and the tape, such as interfacial adhesion energy and contact angles. By combining different sets of timer geometry, ink, and tape, tunable flow delays on paper can be created in a scalable manner from tens of seconds to minutes and even hours, as desired.

### Results

### Tunable Flow Delay Through Delamination

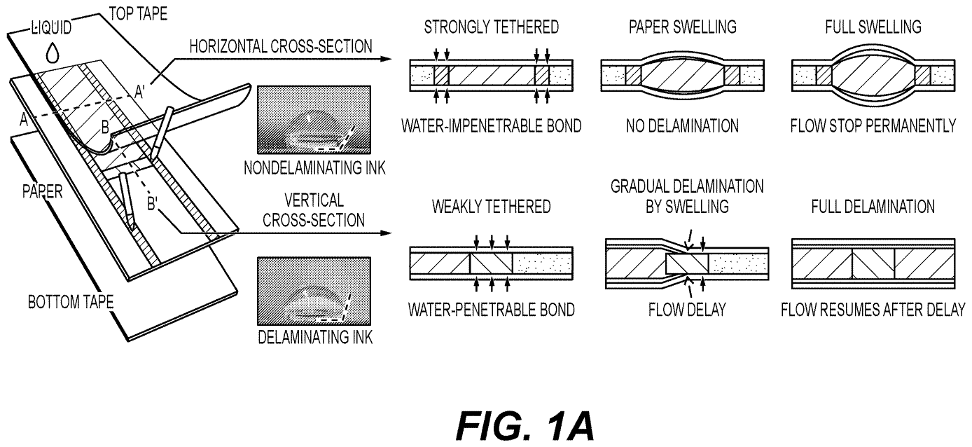

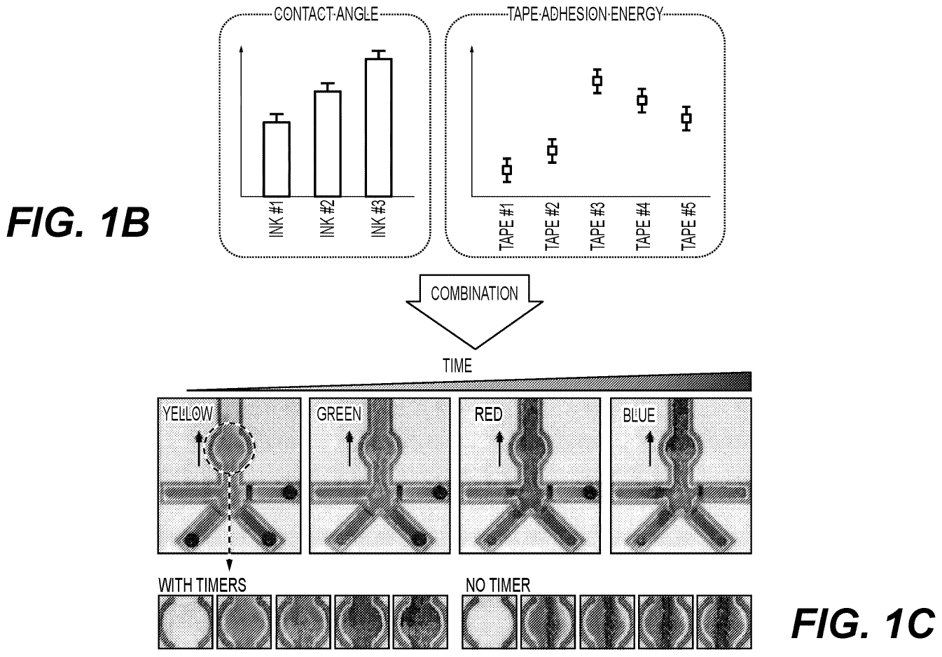

To create tunable flow delay on laminated paper, the paper with two types of strategically selected water-insoluble inks can be patterned in which: non-delaminating and delaminating ink which present different wetting responses (FIG. 1A). The non-delaminating ink created water-impenetrable bonds on the drawn region, permanently tethering the ink-infused paper to the tape even when wetted, and hence was used to define the flow channel boundaries. The delaminating ink created water-penetrable weak bonds on the drawn region, eventually delaminating the ink-infused paper from the tape when wetted, and was used to create a delaminating timer that can produce tunable flow delays.

When introducing a liquid to a paper channel equipped with the timer imprinted as a discrete line normal to flow direction, the paper in the channel was wetted and swelled by liquid [25], but the channel boundaries (represented as brown color) clamped the tape and the paper at the boundaries with no delamination and liquid leakage (A-A′, horizontal cross-section). On the other hand, swelling of wetted paper caused gradual delamination of the delaminating ink-infused paper from the tape (represented as green color), forming local voids as the only path for the flow to proceed (B-B′, vertical cross-section), which produced tunable flow delays according to the timer geometry and material properties of the ink and the tape.

By combining a set of the delaminating inks with different levels of hydrophobicity (i.e., contact angle) and the laminating tapes with varying adhesion energy to the ink-infused paper (FIG. 1i), tunable capillary flow delays on paper can be generated within same timer geometry. As a test platform for demonstration, a simple flow layout was designed where four inlet branches merged into a circular channel (FIG. 1C) and simultaneously introduced four distinctly dye solutions to each inlet. Each branch was equipped with the same width of a timer imprinted with different colored delaminating inks (green, red, and black) to differentially delay the flow between the branches. Observed color patterns showed the sequential arrival of four dye solutions to the circular channel, the flow from the branch with no timer arriving the first (yellow) and from the branch with the black timer arriving the last (blue). In a control layout otherwise identical with no timers in all branches, all dye solutions arrived at the circular channel simultaneously, verifying the role of timers in achieving tunable flow delays.

### Mechanism of the Delaminating Timer

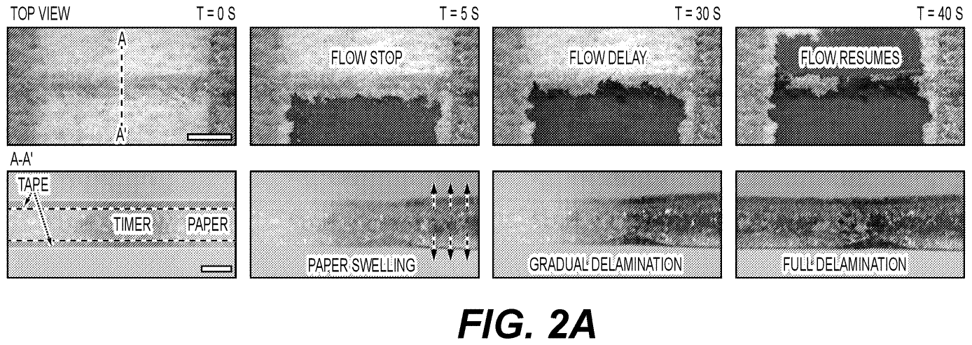

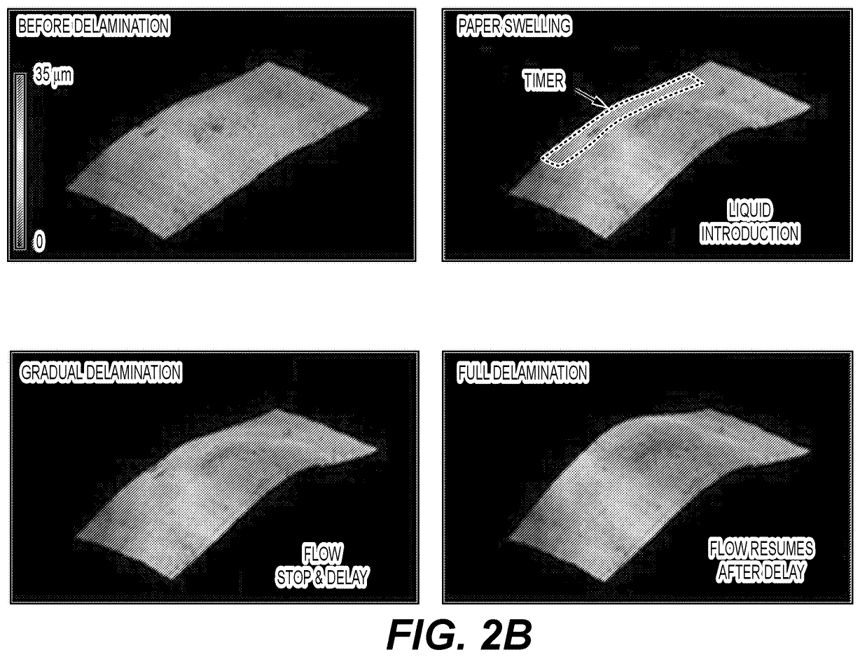

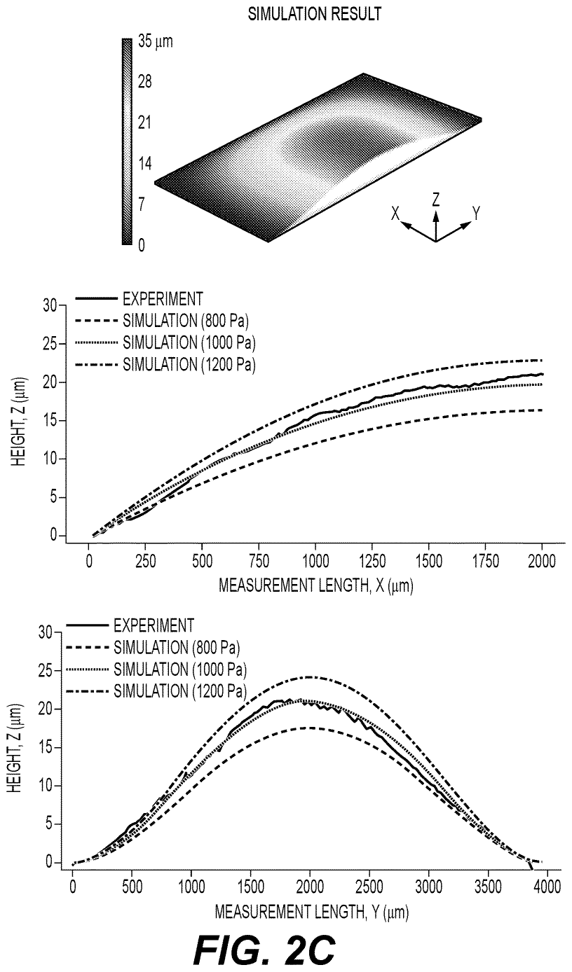

To investigate the mechanism of flow delay on timers through the delamination, the study observed the progress of the dye solution in a flow channel equipped with the timer (FIG. 2A). In a top view, the capillary flow was guided by brown channel boundaries, and a green-colored timer was imprinted as a discrete line normal to flow direction. In a cross-sectional view (A-A′), the timer-equipped channel was laminated with tape on both the top and bottom sides. When the dye solution reached the timer (T=5 s), the flow temporarily stopped at the timer, and the wetted paper started to swell. The swelling of wetted paper at the front of the timer derived the force to gradually delaminate the tape from the ink-infused paper and apply the pressure to proceed with the flow through the local voids formed by the delamination. Until the flow reached the next paper channel, the capillary flow was delayed by this process (T =30 s) and resumed when it reached the next paper channel (T=40 s).

To verify the swelling-induced delamination, surface profiles were measured with height change at the corresponding timepoints (FIG. 2B). Before delamination, there was no significant difference in height across the measuring surface. When introducing liquid to the channel, the wetted paper at the front of the timer swelled to −20 μm, and it gradually propagated to the timer area. When the timer was fully delaminated, the flow resumed, proceeding to the next channel, and all areas swelled to −20 μm, while the channel boundaries remained to be clamped with no height changes. The study calculated the amount of pressure generated by swelling using computer simulation (see Material and Methods) (FIG. 2C). Different amounts of pressure (800-1200 Pa) were applied to a simulation model set in the same condition as the experiment, and the simulation result was compared with that of the experiment. It was found that the simulation of the surface profile on the x and y-axis became almost identical to the experimental measurements when 1000 Pa was applied to the simulation model.

### Modeling of the Delaminating Timer

The calculation of swelling pressure allowed us to attempt to model the tunable flow delay through the delaminating timer. The study employed an equation for surface energy change ΔEsurface when a thin film (i.e., laminated tape) is delaminated from a substrate (i.e., ink-infused paper) by a peeling force at a 90° angle in a liquid environment (see Material and Methods) [26]. This equation can then be combined with Darcy's law using the hydraulic power formula, which allowed derivation of the model for the tunable delay as

$${t}_{d} = ( \frac{( {G}_{tp} - {γ}_{l} ( cos {θ}_{t} + cos {θ}_{p} ) ) ( 1 - ρ ) b μ}{{kP}^{2} A} ) {l}^{2}$$

where t_{d} is the flow delay (in s), Gₜₚ is the interfacial adhesion energy between the tape and the ink-infused paper in a dry air condition, γₗ is liquid surface tension, θₜ and θₚ are the contact angle of the liquid on the tape and the ink-infused paper, respectively, ρ is the porosity of the paper, b is the channel wideness, μ is the dynamic viscosity of the liquid, k is the permeability of the local void formed by swelling, P is the pressure, A is the cross-sectional area generated by swelling, and 1 is the timer width (in mm). The derived model implicates that the capillary flow delay can be tuned either by modifying the timer width l or combining different sets of the tape and the delaminating ink presenting varying Gₜₛ, θₜ, and θₚ.

### Characterization of the Timer on Delaminating Ink and Geometry

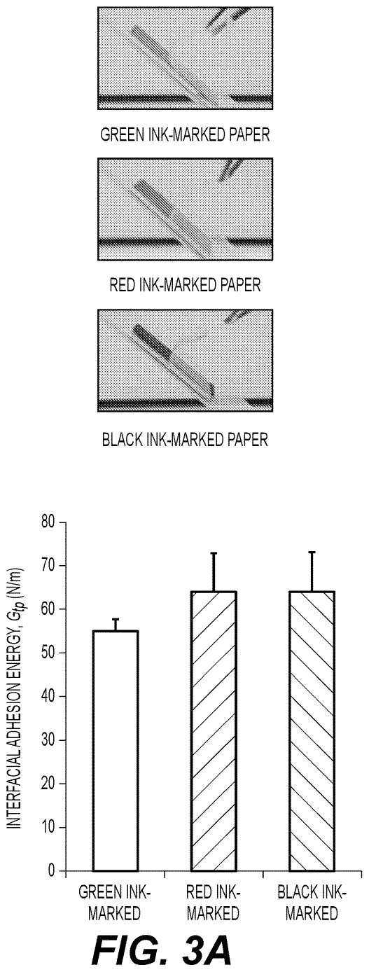

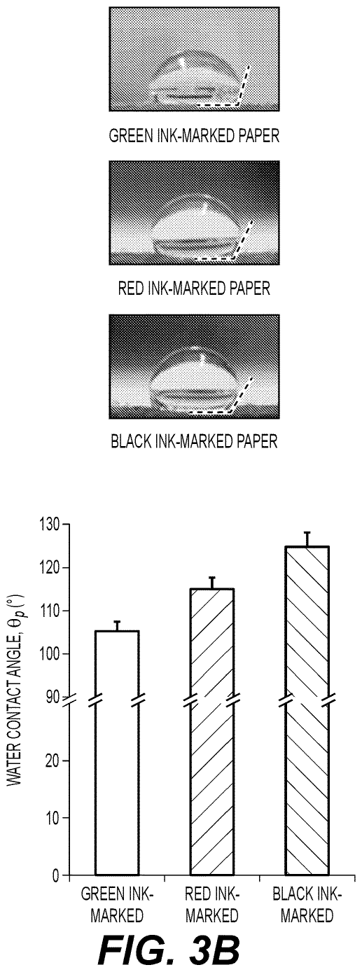

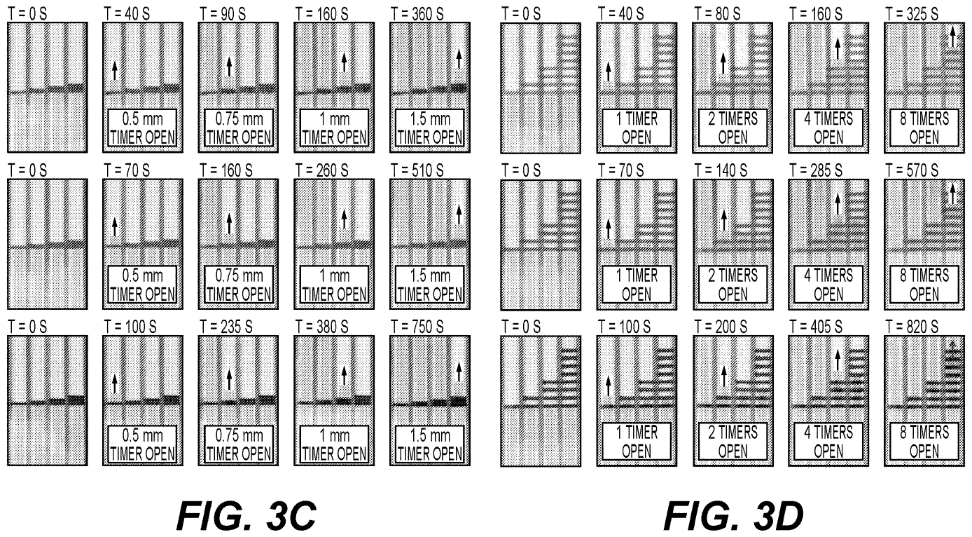

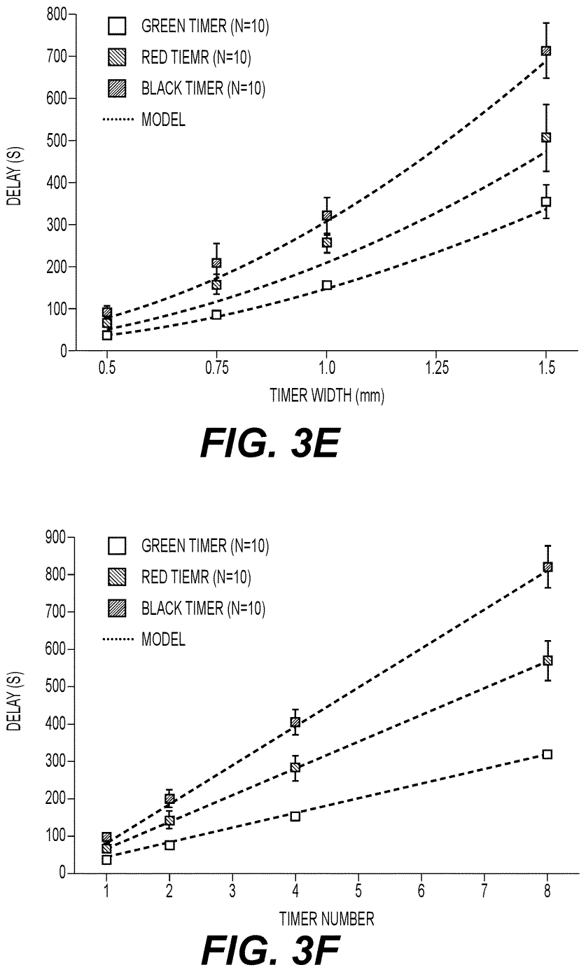

On the basis of the model, the study experimentally characterized the delay on different types of delaminating ink with varying Gₜₚ and θₚ values and the delay on the timer geometries (width and number). The study identified three delaminating inks (green, red, and black) among commercially available permanent markers with different Gₜₚ (FIG. 3A) and θₚ (FIG. 3B) on ink-infused paper. These values were experimentally measured with the same laminating tape (see Material and Methods). The Gₜₚ and θₚvalues on green, red, and black ink-infused paper were measured as ~55, 64, 64 N/m and ~106, 115, 125°, respectively. Those inks were also used to create different geometries of the timers on the channel.

The amount of delay produced by three colored timers with increased widths (0.5 to 3 mm) was measured in independent experiments by observing the progress of dye solutions through these timers (FIG. 3C). It was found that the flow delays in all colored timers nonlinearly (quadratic relation) increased with their width, and the timer imprinted with the more hydrophobic ink produced longer delays (black >red >green) within the same geometry (FIG. 3D). These trends were anticipated according to the derived model as the flow delay relates to the square of the width l2 and the contact angle θp. The derived model accurately expected the flow delays in all measurements. The study also evaluated cascaded timer arrangements to achieve linear control and less variation over the flow delay by placing multiple timers (1 to 8 timers) imprinted with 0.5 mm width (FIG. 3E). The study confirmed that the total delay in all settings of three colored timers was linearly increased with the number of timers (FIG. 3F), and the delay variation was less than that from width-based timers, likely because the noise in the delay can be increased in wider timer due to higher entropy in the delamination process [5]. Together, the study concluded that the derived model can accurately expect the flow delay, and both the geometries (width and number) and material properties (Gₜₚ and θₚ) are the parameters that can easily be tunable to set desired time delays for capillary flow on paper.

### Characterization of the Timer on Laminating Tapes

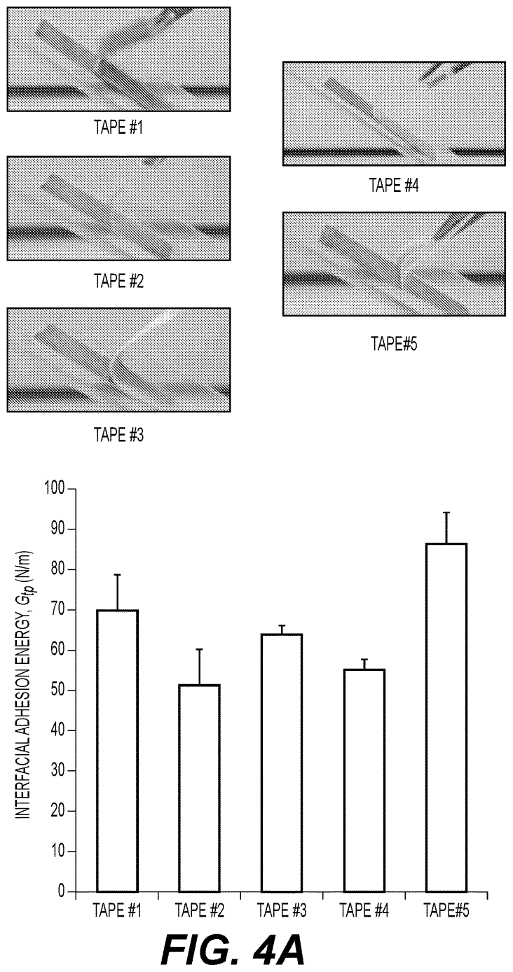

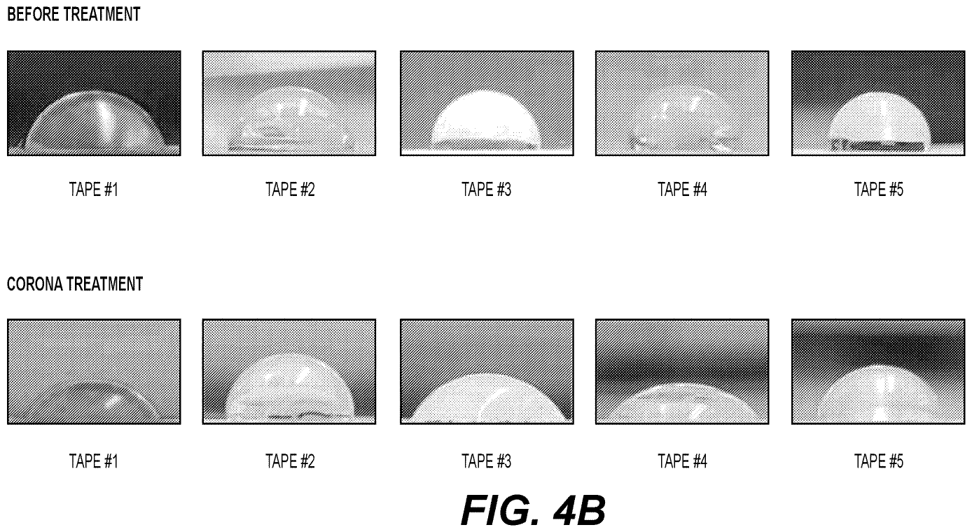

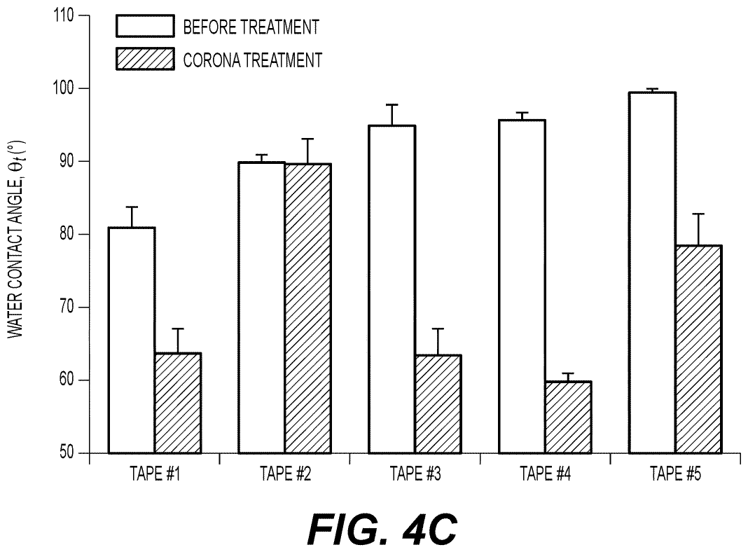

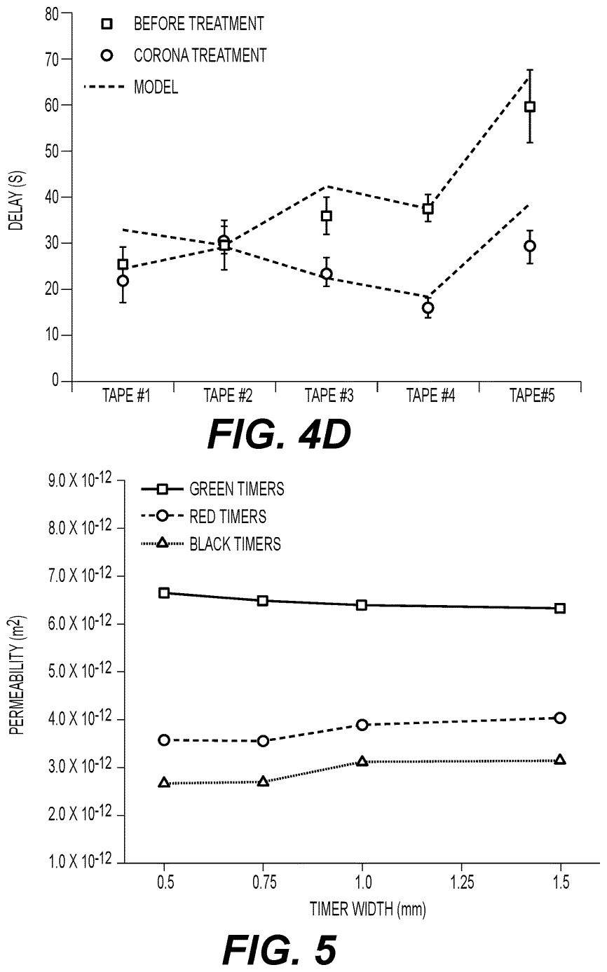

Next, the study characterized the delay on different laminating tapes. The study strategically chose 5 different adhesive tapes with varying Gₜₚ (FIG. 4A) and θₜ values. The adhesive side of these tapes was also treated with a corona discharge to make the tape surface hydrophilic (FIG. 4B). The green delaminating ink was used for all measurements. Before corona treatment, the surface of Tape #1 on the adhesive side was hydrophilic (1°), but all other tapes were hydrophobic (≥90°). After corona treatment for 1 min, all tested tapes were converted to hydrophilic except for Tape #2, which was not affected by corona treatment, and its θₜ remained the same (~90°) (FIG. 4C). The study did not observe any noticeable change in Gₜₚ values from all tested tapes after the corona treatment. Those tapes before/after the corona treatment were used to experimentally measure the flow delay in channels equipped with a 0.5 mm-wide green timer. The measured Gₜₚ and θₜ before/after the corona treatment was applied to the derived model to expect the delay. As anticipated, the measured flow delay produced by the timer decreased after corona treatment except for Tape #2 (FIG. 4D) as the hydrophilic tape surface makes water easier to proceed through local voids formed by the delamination. Furthermore, the derived model could predict the flow delay produced by the timer laminated with the tested tapes and also even when the surfaces of those tapes were treated with the corona discharge.

### DISCUSSION

The exemplary system and method employ an intrinsic mechanism to achieve tunable flow delay on the paper by imprinting patterns with water-insoluble inks. Swelling of wetted paper derived the gradual delamination of ink-infused paper (i.e., delaminating timer) from the tape and thereby delay the capillary flow until the timer is fully delaminated. Combinations of different inks and tapes could selectively tune the material properties of the timer, interfacial adhesion energy, and the contact angle), which produce the tunable flow delays on paper as expected from the derived model. With the expansion of available materials, it is envision that the fluidic circuitry implemented on paper can be used to make complex and traditionally labor-intensive assays available in simple paper formats. Considering how common and readily available the paper is, this method has the potential to be widely accepted in the fields of sensor applications for environmental/food safety and biomedicine for diagnostic, therapeutic, and research purposes.

### Materials and Methods

Fabrication of Timer-Imprinted Channel The fluidic channel equipped with timers was fabricated by drawing lines on tissue paper (Kimtech Science Kimwipes delicate task wipers, Kimberly-Clark, Irving, TX) with two types of water-insoluble inks: non-delaminating and delaminating ink. The nondelaminating ink was used to define channel boundaries to guide the capillary flow. Three different colors (green, red, and black) of delaminating inks with different hydrophobicity were used to create timers with varying delays. Before drawing the lines, one side of the tissue paper was laminated with one of 5 tested tapes. The drawing was conducted using an automatic drawing machine (Silhouette CAMEO, Silhouette America, Lindon, Utah, TX) to precisely control the line geometry. After patterning all lines, the remaining side of the paper was covered with the same tape with holes punched as solution inlets.

### Water-Insoluble Inks and Laminating Tapes

A Sharpie metallic permanent marker was used as the nondelaminating ink to define channel boundaries. Green, red, and black colors of Sharpie ultrafine point marker were used as the delaminating inks to create timers with varying flow delays. One of 5 tapes was used as laminating tapes: tape #1: Fisherbrand Cleanroom Vinyl Tape (Fisher Scientific, NH, USA), tape #2: Scotch Permanent Double Sided Tape (3M Scotch, MN, USA), tape #3: Rainbow Laboratory Tape (Nev's Ink, Inc., WI, USA), tape #4: Scotch Heavy Duty Shipping Packaging Tape, and tape #5: Scotch Magic Tape. All experiments in FIG. 2A-2C and FIG. 3A-3F were performed with tape #4 as laminating tape, and all types of tape were tested in FIG. 4A-4D.

Computer Simulation

The computer simulation was conducted using COMSOL Multiphysics v5.6 (COMSOL, MA, USA) to calculate the swelling pressure. Solid mechanics (solid) was used as the main physics with 40 μm of acrylic plastic as laminating tape backing material (density: 1190 kg/m3, Young's modulus: 2.7e9 Pa, Poisson's ratio: 0.35). Different amounts of pressure (800 to 1200 Pa) were applied to the tape surface to simulate swelling, and the changes in surface profile were calculated on the x and y-axis. The simulated surface profile was then compared with that measured experimentally using Keyence VK-X3000 3D Surface Profiler.

### Modeling of the Delaminating Timer

To derive the model of the flow delay on the delaminating timer, the study first employed an equation for surface energy change ΔEsurface when a laminating tape delaminates from the ink-infused paper by a peeling force at a 90° angle in a liquid environment.

$$Δ {E}_{surface} = ( {G}_{t p} - γ ( cos {θ}_{t} + cos {θ}_{p} ) ) ( 1 - ρ ) b l$$

where Gₜₚ is the interfacial adhesion energy between the tape and the ink-infused paper in a dry air condition, γₗ is liquid surface tension, θₜ and θₚ are the contact angle of the liquid on the tape and the ink-infused paper, respectively, ρ is the porosity of the paper, b is the channel wideness, and 1 is the timer width (in mm). Then, this equation was coupled with Darcy's law using the hydraulic power formula.

$$Q = k \frac{PA}{μ l}$$

$$Power = Q * P = k \frac{{P}^{2} A}{μ l}$$

$$Δ {E}_{surface} = Power * {t}_{d}$$

where Q is the volumetric flow rate, P is the pressure difference across the delaminating timer (i.e., swelling pressure), k is the permeability of the timer, A is the cross-sectional area generated by swelling, μ is the dynamic viscosity of liquid, and t_{d} is the flow delay (in s). The coupling of these two equations allowed us to derive the following model of flow delay on the delaminating timer as

$${t}_{d} = ( \frac{( {G}_{tp} - {γ}_{l} ( cos {θ}_{t} + cos {θ}_{p} ) ) ( 1 - ρ ) b μ}{{kP}^{2} A} ) {l}^{2}$$

The Gₜₚ values were measured experimentally by peeling off the laminated tape from the ink-infused papers using a TMI Lab Master Release & Adhesion Tester. The θₚ values were measured by applying water droplets onto the surface of the ink-infused papers. γₗ=72 mN/m, ρ=0.62, b=4 (mm), μ=1 mPa·s, and A=8.72×10⁻² (mm)² (calculated from surface profile result). The k of three colored timers were also experimentally measured: green: 6.43 μm², red: 5.34 μm², and black: 3.65 μm².

### Permeability of the Different Colored Timer

The local voids which was formed by the delamination at the interface between the timer and the laminating tape were considered as porous media. The permeability k of the delaminating timers was experimentally measured by observing the progress of dyed solution through these timers with different widths (FIG. 5) using the following equation as

$$k = v \frac{μ l}{P}$$

where ν is the average velocity of fluid passing through the delaminating timer.

The average of the measured permeability in each colored timer was used in the derived model.

### REFERENCES

1. E. K. Sackmann, A. L. Fulton, D. J. Beebe, The present and future role of microfluidics in biomedical research. Nature. 507, 181-189 (2014).
2. M. M. Gong, D. Sinton, Turning the page: Advancing paper-based microfluidics for broad diagnostic application. Chem. Rev. 117, 8447-8480 (2017).
3. K. Yamada, H. Shibata, K. Suzuki, D. Citterio, Toward practical application of paper-based microfluidics for medical diagnostics: state-of-the-art and challenges. Lab Chip. 17, 1206-1249 (2017).
4. A. K. Yetisen, M. S. Akram, C. R. Lowe, Paper-based microfluidic point-of-care diagnostic devices. Lab Chip. 13, 2210-2251 (2013).
5. D. Lee, T. Ozkaya-Ahmadov, C.-H. Chu, M. Boya, R. Liu, A. F. Sarioglu, Capillary flow control in lateral flow assays via delaminating timers. Sci. Adv. 7, eabf9833 (2021).
6. H. Oh, H. Ahn, A. Tripathi, A closer look into FDA-EUA approved diagnostic techniques of Covid-19. ACS Infect. Dis. 7, 2787-2800 (2021).
7. A. Biby, X. Wang, X. Liu, O. Roberson, A. Henry, X. Xia, Rapid testing for coronavirus disease 2019 (COVID-19). MRS Communications. 12, 12-23 (2022).
8. A. W. Martinez, S. T. Phillips, M. J. Butte, G. M. Whitesides, Patterned paper as a platform for inexpensive, low-volume, portable bioassays. Angew. Chemie—Int. Ed. 46, 1318-1320 (2007).
9. A. W. Martinez, S. T. Phillips, G. M. Whitesides, Three-dimensional microfluidic devices fabricated in layered paper and tape. Proc. Natl. Acad. Sci. U.S.A 105, 19606-19611 (2008).
10. C.-M. Cheng, A. W. Martinez, J. Gong, C. R. Mace, S. T. Phillips, E. Carrilho, K. A. Mirica, G. M. Whitesides, Paper-based ELISA. Angew. Chemie-Int. Ed. 49, 4771-4774 (2010).
11. B. J. Toley, B. McKenzie, T. Liang, J. R. Buser, P. Yager, E. Fu, Tunable-Delay Shunts for Paper Microfluidic Devices. Anal. Chem. 85, 11545-11552 (2013).
12. E. Fu, T. Liang, P. Spicar-Mihalic, J. Houghtaling, S. Ramachandran, P. Yager, Two-dimensional paper network format that enables simple multistep assays for use in low-resource settings in the context of malaria antigen detection. Anal. Chem. 84, 4574-4579 (2012).
13. R. Banerjee, A. Jaiswal, Recent advances in nanoparticle-based lateral flow immunoassay as a point-of-care diagnostic tool for infectious agents and diseases. Analyst. 143, 1970-1996 (2018).
14. D. M. Cate, J. A. Adkins, J. Mettakoonpitak, C. S. Henry, Recent developments in paper-based microfluidic devices. Anal. Chem. 87, 19-41 (2015).
15. Y. Yang, E. Noviana, M. P. Nguyen, B. J. Geiss, D. S. Dandy, C. S. Henry, Paper-based microfluidic devices: Emerging themes and applications. Anal. Chem. 89, 71-91 (2017).
16. N. Raj, V. Breedveld, D. W. Hess, Flow control in fully enclosed microfluidics paper based analytical devices using plasma processes. Sens. Actuators B: Chem. 320, 128606 (2020).
17. I. Jang, S. Song, Facile and precise flow control for a paper-based microfluidic device through varying paper permeability. Lab Chip. 15, 3405-3412 (2015).
18. E. Fu, B. Lutz, P. Kauffman, P. Yager, Controlled reagent transport in disposable 2D paper networks. Lab Chip. 10, 918-920 (2010).
19. J. H. Shin, J. Park, S. H. Kim, J. K. Park, Programmed sample delivery on a pressurized paper. Biomicrofluidics. 8, 0541211 (2014).
20. B. Lutz, T. Liang, E. Fu, S. Ramachandran, P. Kauffman, P. Yager, Dissolvable fluidic time delays for programming multi-step assays in instrument-free paper diagnostics. Lab Chip. 13, 2840-2847 (2013).
21. H. Chen, J. Cogswell, C. Anagnostopoulos, M. Faghri, A fluidic diode, valves, and a sequential-loading circuit fabricated on layered paper. Lab Chip. 12, 2909-2913 (2012).
22. X. Li, P. Zwanenburg, X. Liu, Magnetic timing valves for fluid control in paper-based microfluidics. Lab Chip. 13, 2609-2614 (2013).
23. M. Fratzl, B. S. Chang, S. Oyola-Reynoso, G. Blaire, S. Delshadi, T. Devillers, T. Ward, N. M. Dempsey, J.-F. Bloch, M. M. Thuo, Magnetic Two-Way Valves for Paper-Based Capillary-Driven Microfluidic Devices. ACS Omega. 3, 2049-2057 (2018).
24. H. Fu, P. Song, Q. Wu, C. Zhao, P. Pan, X. Li, N. Y. K. Li-Jessen, X. Liu, A paper-based microfluidic platform with shapememory-polymer-actuated fluid valves for automated multi-step immunoassays. Microsyst. Nanoeng. 5, 50 (2019).
25. M. Y. Hashim, M. N. Roslan, A. M. Amin, A. M. A. Zaidi, S. Ariffin, Mercerization treatment parameter effect on natural fiber reinforced polymer matrix composite: A brief review. World Acad. Sci. Eng. Technol. 6, 1382-1388 (2012).
26. J. K. Park, Y. Zhang, B. Xu, S. Kim, Pattern transfer of large-scale thin membranes with controllable self-delamination interface for integrated functional systems. Nat. Commun. 12, 6882 (2020).

### Example 2. Chemically Amplified Multiplex Detection of SARS-CoV-2, Influenza A And B Viruses Via Paint-Programmed Lateral Flow Assays

Severe acute respiratory syndrome-coronavirus 2 (SARS-CoV-2) continues to threaten the lives by evolving into new variants with greater transmissibility. Although lateral flow assays (LFAs) are widely used to self-test for coronavirus disease 2019 (COVID-19), these tests suffer from low sensitivity, leading to a high rate of false negative results. A study was conducted to develop and evaluate multiplexed lateral flow assay for the detection of SARS-CoV-2, influenza A, and B viruses in human saliva with a built-in chemical amplification of colorimetric signal for enhanced sensitivity. To automate the amplification process, the paper-based device was integrated with an imprinted flow controller, which coordinates the routing of different reagents and ensures their sequential and timely delivery to run an optimal amplification reaction. Using the assay, the study could detect SARS-CoV-2 and influenza A and B viruses with ~25× higher sensitivity than commercial LFAs and showed that the device can detect SARS-CoV-2-positive patient saliva samples missed by commercial LFAs. The exemplary method and system provide an effective and practical solution to enhance the performance of conventional LFAs and will enable sensitive self-testing to prevent virus transmission and future outbreaks of new variants.

### INTRODUCTION

Coronavirus disease 2019 (COVID-19), declared a pandemic in March 2020 by the World Health Organization, is now transforming into an infectious disease inseparable from the daily life.1-5 Despite emergent vaccine development and global efforts in managing the COVID-19 crisis, the virus is still spreading around the globe and remains a problem threatening lives by evolving into new variants such as delta or omicron.⁵⁻⁸ The most recent variant BA.⁵, the highly contagious omicron subvariant, can evade vaccines by escaping from neutralizing antibodies,⁸⁻¹¹ and is responsible for >88% of new COVID-19 cases across the U.S. (data obtained on 27th August 2022 from the U.S. Centers for Disease Control and Prevention). The efficient transmission of these new variants is also due to the fact that the infections are often asymptomatic or with mild symptoms that may easily be confused with common flu.¹²⁻¹⁵ Considering that ~35% of the cases have no symptoms and these asymptomatic cases are responsible for ~59% of virus transmission,¹⁶⁻¹⁹ pointing to an urgent need to have COVID-19 self-testing that can discriminate between SARS-CoV-2 infections and common flu while being widely accessible for anyone anywhere.

The gold standard platform for self-testing has long been the lateral flow assays (LFAs).20-23 LFAs are paper-based diagnostic devices that can run simple assays at home or in point-of-care (POC) settings. Their portability, low cost, ease of use, compatibility with various bodily fluids, and ability to provide results rapidly make LFAs the most common diagnostic platform for COVID-19 self-testing.²³⁻²⁵ LFA-based COVID-19 testing can generally be divided into two types: antibody and antigen tests.²³⁻²⁵ Antibody tests, known as serological tests, are typically targeting immunoglobulin M and G antibodies which are expressed in blood or saliva against virus infection. Although antibody tests can be used to study the immune response to the virus or to determine the precise rate of infection and seroconversion, they usually cannot help to diagnose current acute infection as it takes 1-2 weeks post symptom onset for patients to develop antibodies in the body, i.e., detect past infections.²⁶⁻²⁸ Antigen tests diagnosing active acute infections are more advantageous for tracking and real-time monitoring of highly contagious viruses than antibody tests.²⁸⁻³⁰ Antigen tests typically detect nucleocapsid protein (NP) coded by the N gene, the most conserved among mutant strains during spread and evolution.³¹⁻³³ Despite its advantages, the inherent low sensitivity of the test²²⁻³⁰ compels commercial LFAs to necessitate invasive swabs, such as nasopharyngeal or nasal swabs, instead of using non-invasive specimens (e.g., saliva). Beyond the unpleasant experience, required more invasive sampling leads to assay reluctance and results in less frequent self-testing, ultimately defeating the purpose of identifying infections early on to minimize transmission.

A study was conducted that developed an exemplary LFA that runs an internal amplification reaction while screening a saliva sample simultaneously for SARS-CoV-2 and influenza A and B viruses for higher sensitivity and specificity. The signal amplification process within the exemplary LFA is automated by a paint-programmed²²⁻³⁵ flow controller that orchestrates the capillary flow of different reagents across the paper-based device. Inside the device, flow paths equipped with delaminating timer gates are set to open at pre-programmed time points, which ensures sequential delivery of reagents with optimal incubation times in between while running an autometallographic reaction to enhance the colorimetric signals from the LFA. The study characterized the assay with simulated saliva samples spiked with known amounts of SARS-CoV-2 and influenza A and B viruses and compared them with commercially available LFAs. Finally, the study employed the technology to screen samples collected from patients with known COVID-19 infections to investigate their clinical feasibility.

### Results

### Device Design and Operation

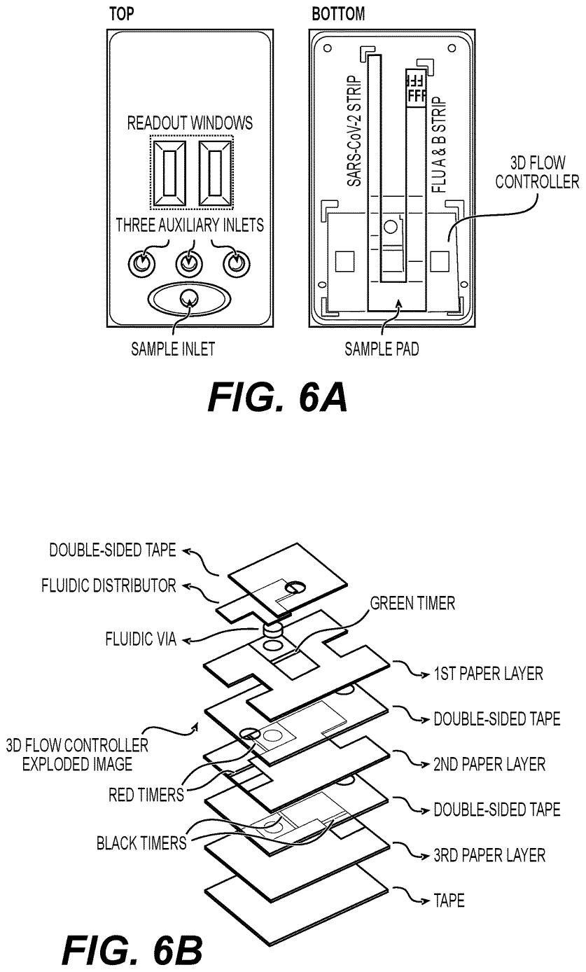

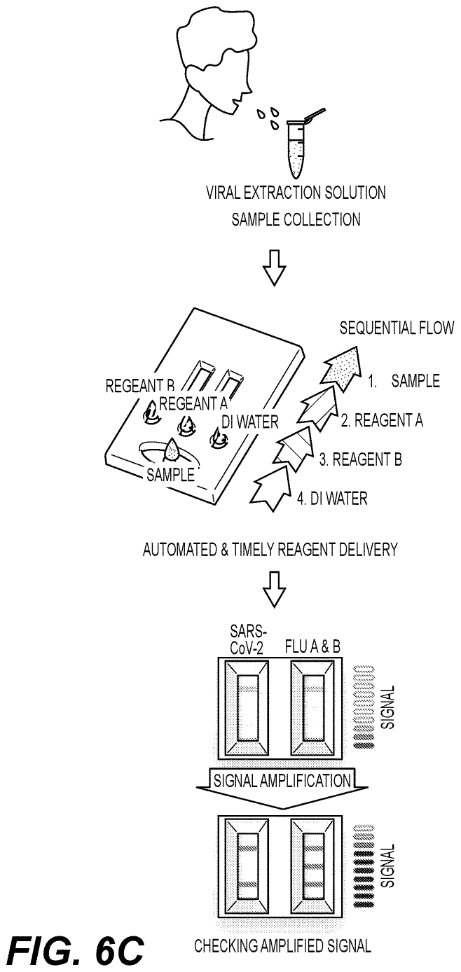

The assay consisted of two different commercial LFA test strips with SARS-CoV-2 and influenza A & B virus-specific recognition antibodies immobilized on the corresponding test lines and a paper-based flow controller that was custom-programmed to execute the colorimetric signal amplification reaction on those test lines (FIG. 6A). The two components were then placed within a 3D-printed case, where the sample and the auxiliary reagent inlets were coupled with the flow controller and the test lines were exposed for reading the colorimetric results.

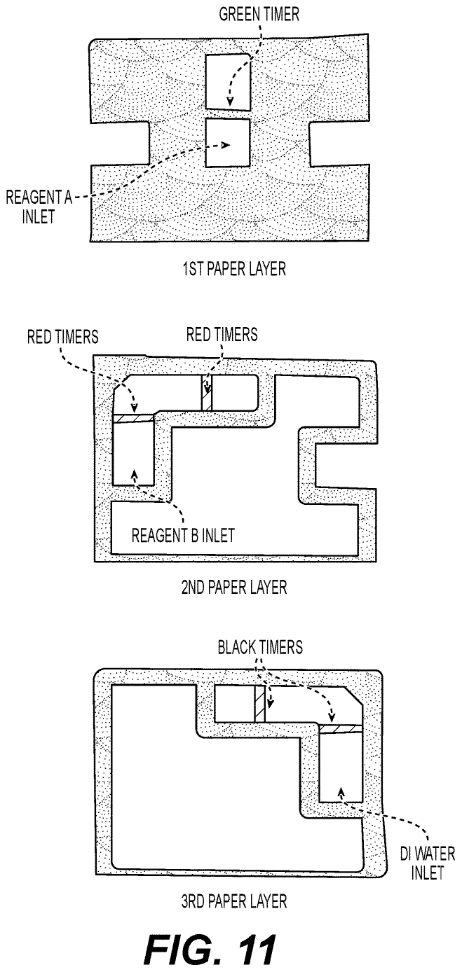

The study designed the flow controller as a multi-layered paper-based system, in which adhesive polymer tapes were used to bond individual paper layers and also laminate the whole device (FIG. 6B). Each paper layer was designed to carry a specific reagent from the corresponding inlet into the reaction spot (i.e., the test lines) at the desired time. Differential routing in paper layers was achieved by imprinting distinct flow paths with combinations of water-insoluble inks that differentially modified the adhesion between the polymer tape and wetted paper (FIG. 11). Specifically, the ink (brown-colored) used to outline the flow paths permanently adhered to the polymer tapes, while the inks used as roadblocks on the flow paths had a weaker affinity to the polymer tape when wetted and eventually delaminated to create a bypass flow path for the flow to resume.^{22,35} In the design, the study used inks with different affinities to the polymer tape, which were also chosen with different colors for visualization purposes, to create these temporary roadblocks so that reagents can be delivered to the reaction spots sequentially with set time delays: the green timer in 1st paper layer was set to expire first, followed by the red timer in 2nd layer and then the black timer in 3rd layer (FIG. 6B). Once released reagents in different paper layers were transferred to the reaction layer through fluidic vias (i.e., disc-shaped cellulose paper placed in cutouts in polymer tapes). Within the reaction layer, the reagents were finally distributed to the two LFA strips by a fluidic distributor for amplification.

In the construction of the flow controller, the adhesive polymer tapes are critical components and serve multiple purposes: First, the double-sided adhesive tapes placed in between paper layers bonded all layers together. Second, the polymer tapes fluidically isolated capillary flows in different layers and allowed transfer exclusively through vias formed by cut-outs on tapes. Third, the polymer tapes enabled the operation of delaminating timers, which required the forming gap between the paper and the polymer to open a bypass path to release the capillary flow after a set delay. Finally, the polymer tapes laminated the whole construct from top to bottom to protect it from environmental interference.

The operation of the final device is a straightforward process that can potentially be performed by nonspecialists at home or in POC settings and does not require external instruments for a readout (FIG. 6C). The sample is collected by spitting saliva into a viral extraction solution containing buffer for viral lysis. The sample mixture is applied to the sample inlet, and at the same time, two chemical reagents for signal amplification (reagent A and B) and DI water are loaded to three auxiliary inlets. Note that these amplification reagents and the DI water can potentially be integrated onto the assay in containers that can be released on-demand. The rest of the process is automatically handled by the device, and the results are observed as color changes in the test lines. If the concentration of any of the SARS-CoV-2 and influenza A and B viruses in the saliva sample is more than the detection limit of the commercial assay, red bands are initially observed in the readout window. At the completion of the assay (at −20 mins), the chemically amplified signal is observed as black bands (see the Materials and Methods). Finally, because the assay has a built-in wash step quenching the reaction, it eliminates potential artifacts from delayed observation of the colorimetric results.

### Programmed Capillary Flow Routing for Automated Signal Amplification

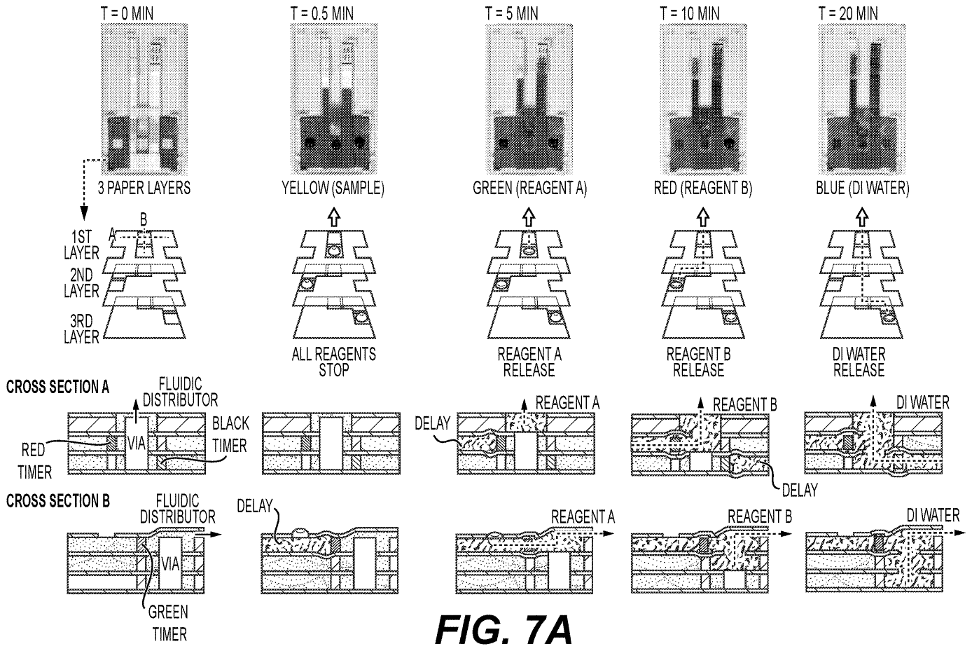

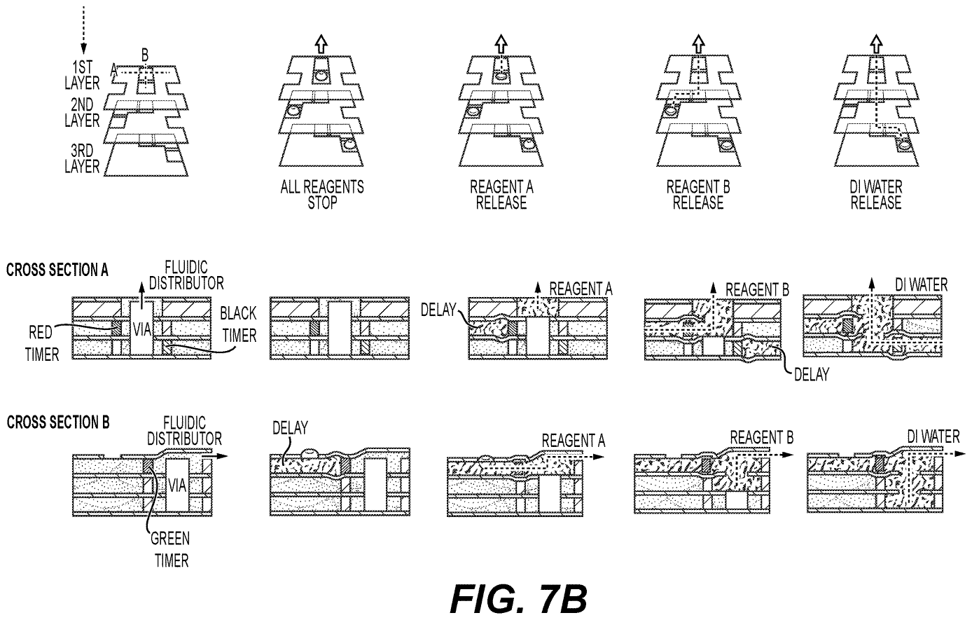

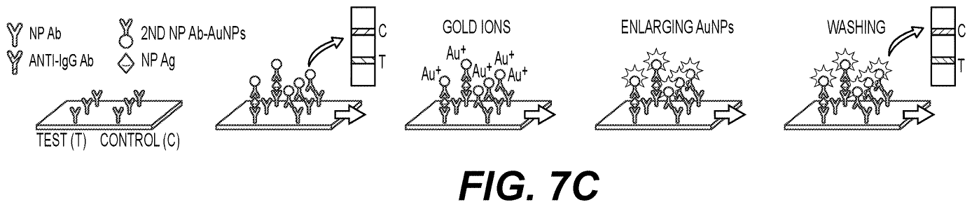

The exemplaryh paper-based flow controller was physically programmed by painting with a palette of water-insoluble inks for automated execution of a multi-step chemical reaction to amplify the colorimetric LFA signals. To tune the timing of delivery for different reagents within the device, the study optimized the operation of the flow controller with dye solutions instead of actual chemicals (FIG. 7A). In the optimized device, the sample and the signal amplification reagents were successfully delivered to the test lines in the desired sequence leaving intended incubation times in between reaction steps. Specifically, the sample (yellow, 60 L) reached the test lines in ~30 s, followed by reagent A (green, 30 μL) from 1st paper layer and reagent B (red, 30 L) from 2nd layer reaching the test lines in 5 and 10 min, respectively. As the final step, DI water (blue, 60 L) reached in ~20 min and washed the test lines to quench the reaction. With the design, the study achieved a total assay time of 20 min, which is similar to the time required for conventional LFAs for COVID-19 testing.

Also matching with the experimental observations, specific routes taken by reagents in each layer of the flow controller, along with the state of timer gates throughout the assay, are illustrated in a schematic (FIG. 7B). At the instant the saliva sample (yellow) reached the test lines, all reagents were still roadblocked by timer gates. The green timer expired first and released reagent A (green), which followed a straight path before being lifted to the top reaction layer and the test lines, while reagent B (red) cleared the first red timer but was held by the second red timer. At this point, the DI water (blue) did not yet clear any of the black timers. Next, reagent B cleared the second red timer and was lifted up and distributed to the test lines on the reaction layer while the second black timer was still holding the DI water in 3rd layer. As the last step, the second black timer expired, and DI water was released, lifted up, and delivered to the test lines for washing. From a technological perspective, the exemplary flow controller design of the study demonstrates the compatibility of delaminating timer gates with multilayered paper-based microfluidic designs and its potential for running complex chemical sequences with minimal dead volume overhead.

Through the programmed delivery of different chemical reagents to the test lines, the device automatically performed an autometallographic reaction to enhance the colorimetric signal for higher sensitivity (FIG. 7C). Briefly, in the conventional LFA test strips integrated within the device, antibodies (NP Ab) specific to nucleocapsid protein antigens (NP Ag) and anti-IgG antibody (Anti-IgG Ab) were immobilized on the test and control lines, respectively. LFAs also harbored gold nanoparticles conjugated with secondary NP antibodies (2nd NP Ab-AuNPs). Therefore, with the introduction of the sample, NP Ag, pre-mixed with saliva, formed a complex with the 2nd NP Ab-AuNPs. The complexes and the unbound AuNPs were then captured at the test and control lines, respectively, to produce the initial non-amplified signals. Next, the sequential delivery of the amplification reagents (reagents A and B) supplied gold ions and then reduced them to their elemental state to be deposited on the immunocaptured AuNPs to increase their size. Enlarged AuNPs led to greater color intensity and darkened the original color tone improving contrast, both of which ultimately provided higher sensitivity. Finally, internal washing of the test lines with DI water at a pre-set time quenched amplifying reaction for consistent readings and ensured against false positive readings.

### Characterization of the Device for SARS-CoV-2 Detection

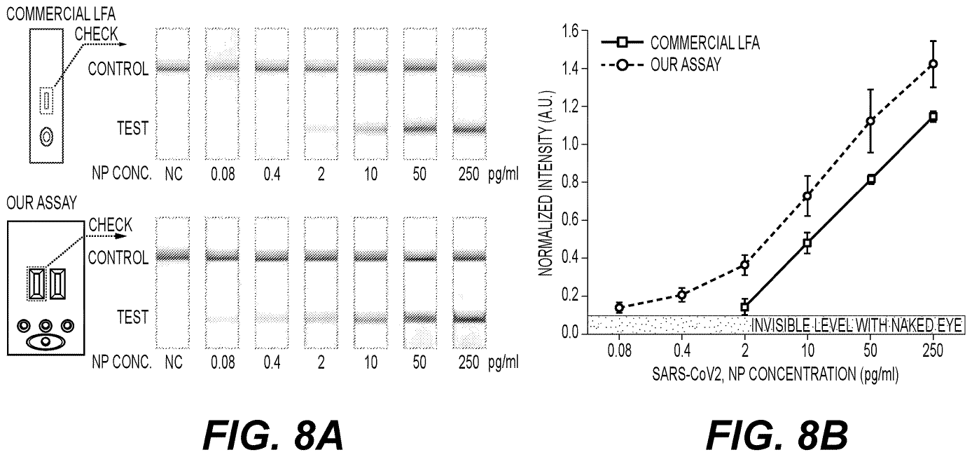

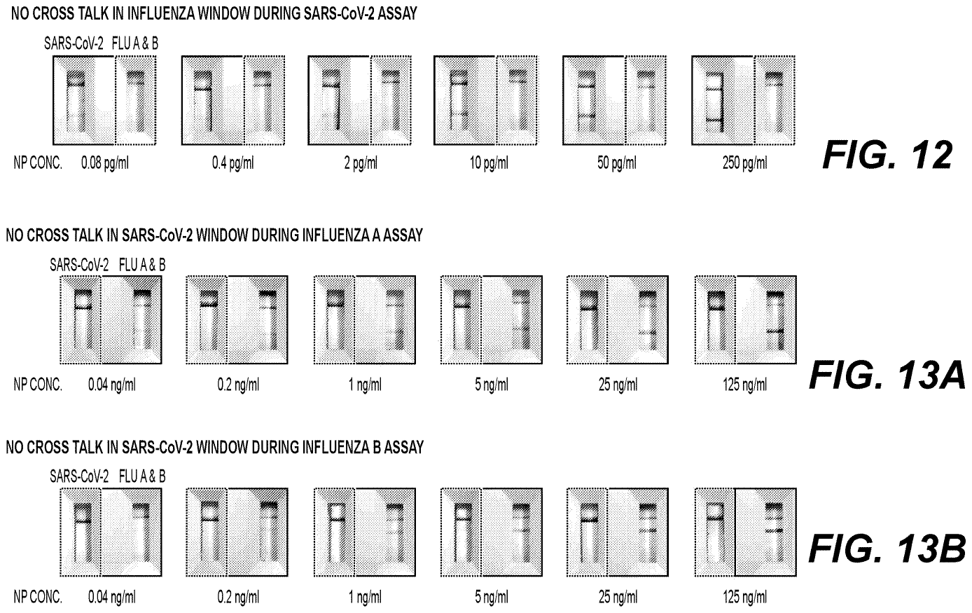

The study first characterized the exemplary device by processing control samples spiked with SARS-CoV-2 NP. In these experiments, commercially obtained recombinant SARS-CoV-2 NP were serially diluted and spiked into saliva samples donated by healthy volunteers to achieve final SARS-CoV-2 NP concentrations ranging from 0.08 to 250 μg/ml. In testing these samples with the device, the study measured the normalized color change of the test line through digital image processing (see Materials and Methods) (FIG. 8A). Based on these measurements, the study attempted to determine the limit of detection (LoD) for the device for SARS-CoV-2 detection. While, the signals with normalized intensity (NI)<0.1 were not visible to the naked eye, the study found that the signals from SARS-CoV-2 NP at concentrations as low as 0.08 pg/ml consistently produced signals with NI>0.1. Meanwhile, no noticeable cross-talk could be observed on the test lines reserved for the Influenza viruses (FIG. 12).

Next, the study benchmarked the assay against the commercial COVID-19 test strip by processing the matched control samples and comparing signals. Among the samples tested, the lowest concentration of SARS-CoV-2 NP that produced a visible signal in the commercial test strip was found to be 2 μg/ml versus the 0.08 pg/ml of the assay (FIG. 8B). In fact, the assay could produce a similar signal level at a concentration of 0.08 pg/ml to that of the commercial test at 2 μg/ml, representing a ~25× enhancement in sensitivity. Remarkably, the assay's ability to detect SARS-CoV-2 NP at concentrations as low as 0.08 pg/ml could be considered comparable to the level of conventional molecular assays such as polymerase chain reaction, because NP concentrations <0.5 pg/ml have been associated with very low virus concentrations (N gene RNA) producing a relatively high cycle threshold value >30.34

### Characterization of the Device for Flu Detection

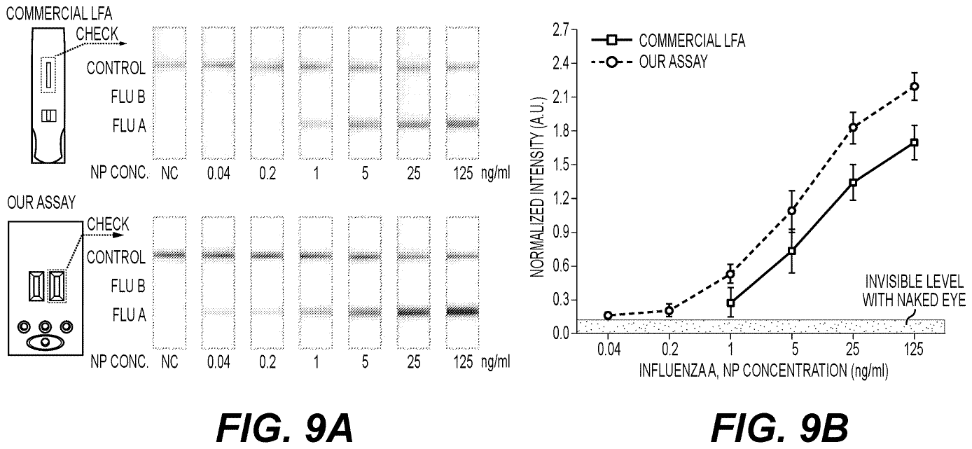

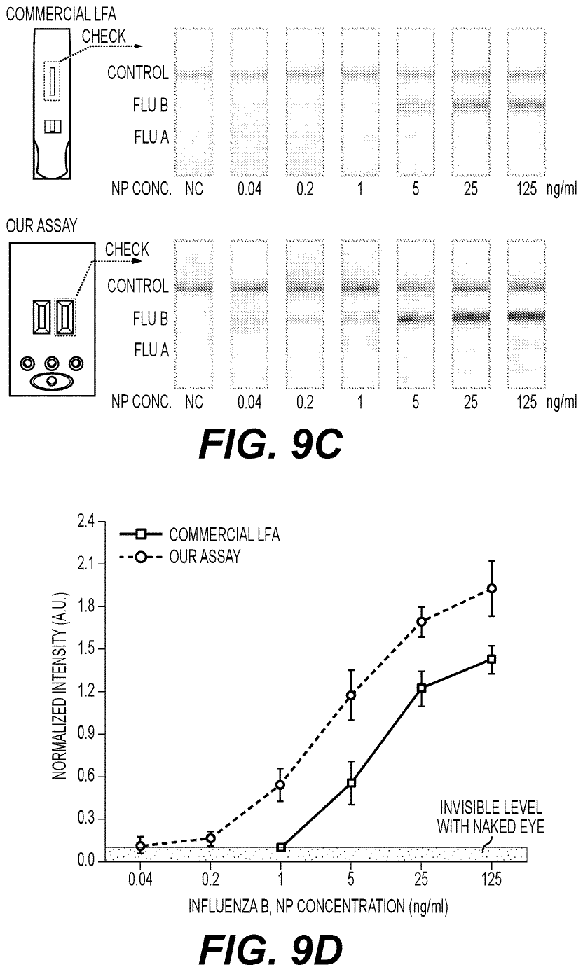

Next, the study investigated the device's sensitivity in detecting flu by processing control samples spiked with influenza A and B viruses. Controlled amounts of recombinant influenza A and B NP were spiked into saliva samples obtained from healthy donors and subsequently transferred to a viral extraction solution. In analyzing these samples with final NP concentrations varying from 0.04 to 125 ng/ml, the study measured the normalized color change of the two test lines corresponding to each virus type through digital image processing (see Materials and Methods) (FIGS. 9A and 9C). In repeated experiments, the device was able to consistently produce visible signals for samples with influenza A NP concentrations as low as 0.04 ng/ml (FIG. 9A). As for the influenza B NP samples, the assay was found to be less sensitive, producing signals visible to the naked eye (i.e., NI>0.1) in ⅔ tests at 0.04 ng/ml (FIG. 9C). In all of the experiments, there was no cross-talk observed between influenza A and B test lines and also with the SARS-CoV-2 test line (FIGS. 13A-13B).

Quantitatively comparing the levels of colorimetric signals from the device with those from the commercial test strip for flu detection, the study found the assay to be more sensitive in detecting influenza A and B virus NPs in matched samples. Not only did the assay produce stronger signals for a given target concentration, but it was also able to detect virus NPs at concentrations of less than the commercial test strip (FIGS. 9B and 9D). Specifically, the signals from the commercial test strip were visible to the naked eye for influenza A samples only for concentrations of 1 ng/ml and higher in contrast with the assay's LoD at 0.04 ng/ml (FIG. 9B). As for the influenza B simulated samples, the minimal concentration of influenza B NP that commercial test could only reliably identify was 5 ng/ml versus 0.2 ng/ml from the assay (FIG. 9D). Taken together, these results demonstrated that the assay could detect influenza A and B viruses at ~25× lower concentrations than the commercial assay due to built-in chemical amplification of the colorimetric signal and also suggest a similar level of sensitivity enhancement to the one observed for the detection of SARS-CoV-2 previously.

Testing Patient Samples with the Developed Technology

To demonstrate the feasibility of employing the technology for the screening of patient samples, the study processed saliva samples of subjects with COVID-19 infection. Patient samples (n=3) were acquired from a vendor who independently confirmed SARS-CoV-2 positivity via RT-qPCR. SARS-CoV-2 negative saliva samples were collected from healthy donors as controls (n=3) under Georgia-Tech Institutional Review Board (IRB)-approved protocol.

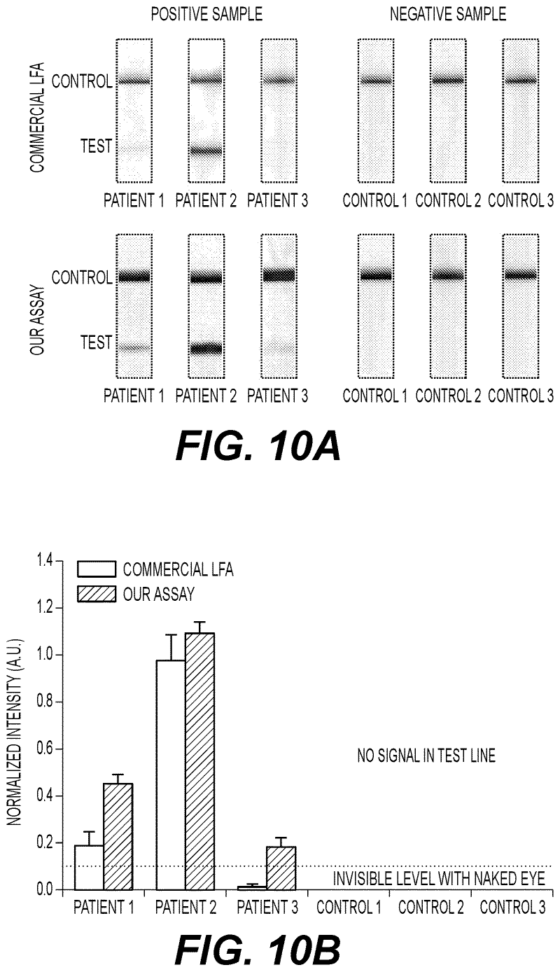

The study first processed the patient samples using the commercial strip. Among the samples the study tested, commercial LFA could only detect SARS-CoV-2 in samples from ⅔ samples (Patients 1 and 2) (FIG. 10A). Among the two correctly identified SARS-CoV-2-positive samples, one (Patient 1) had a faint (NI=~0.19) signal weaker than the other (Patient 2), whose sample produced a high-intensity signal clearly visible to the naked eye (FIG. 10B). However, the remaining sample (Patient 3) did not produce any visible signal at the test line despite a control line signal at a similar level to those identified as positive.

In contrast with the commercial test strip, the exemplary device could successfully detect SARS-CoV-2 in all of the processed patient samples producing visible (NI >0.1) colorimetric signals at the test lines (FIG. 10A). The test line signal, which was absent in the analysis of the false-negative sample (Patient 3) by the commercial strip, was visible (NI=~0.18) in the assay (FIG. 10B). Furthermore, signals already visible in the commercial assay were all amplified with more clear visibility. These amplified results were also specific as none of the processed healthy control samples (n=3) produced signals in the device. Taken together, these results demonstrated not only the capability of the device processing real samples but also highlighted the importance of enhancing the sensitivity of LFAs for pathogen detection to ensure against false negatives as was seen in the experiments.

### DISCUSSION

Asymptomatic cases and a high rate of false negative results because of low assay sensitivity have been the challenges that hampered the effectiveness of LFAs for tracking COVID-19 infections through self-testing. To address both issues, the study developed an LFA that can (1) distinguish COVID-19 from common flu via a multiplexed test panel and (2) detect both SARS-CoV-2 and influenza viruses with a ~25× higher sensitivity than the conventional LFAs due to a built-in chemical amplification. From the outset, the exemplary device operates as simple as conventional LFA wicking fluids spontaneously, while the embedded paper-based flow controller coordinates multiple capillary flows according to a program and automates the optimal execution of the multi-step, multi-reagent chemical amplification of the colorimetric readout. The enhanced sensitivity of the device could help reduce false negative COVID-19 results that are commonplace from the existing tests. Also, considering that the technique can easily be adapted to detect different pathogens, the exemplary system and method has the potential to help manage future outbreaks of new variants.

### Materials and Methods

Fabrication of the custom-designed 3D flow controller

Three paper layers imprinted flow paths and timers were fabricated by drawing lines on tissue paper (Kimtech Science Kimwipes delicate task wipers, Kimberly-Clark, Irving, TX) using an automatic drawing machine (Silhouette CAMEO, Silhouette America, Lindon, Utah, TX).

Before drawing the lines, one side of the tissue paper was laminated with clear tape (Scotch Heavy Duty Shipping Packaging Tape, 3M Scotch, MN, USA). The channel boundaries to guide the capillary flow were patterned on paper using a Sharpie metallic permanent marker (brown), which makes the marked paper permanently tethered to the tape. Three different colored timers producing varying delays were patterned on strategic nodes using Sharpie ultra fine point markers (green, red, and black), where the marked paper eventually delaminates from the tape when wetted producing controlled amounts of delays. The double-sided tapes with holes were then attached for aligning and bonding all three layers. A hole as the location of the fluidic via was punched using a 2.5 mm biopsy punch and filled with the same size of Whatman grade 1 filter paper. The fluidic distributor made of tissue paper was placed on top of the fluidic via, and another double-sided tape was attached to integrate with two commercial LFAs.

### Capillary Flow Control on Paper

To control capillary flow on laminated paper, the exemplary system and method used water-insoluble inks with two different responses to water when wetted, called non-delaminating ink and delaminating ink.^{22,35} The lines drawn with nondelaminating ink (Sharpie metallic permanent marker) are permanently tethered to the tape without delamination from wetting, imprinting capillary flow paths on laminated paper. The lines patterned with three different colors of delaminating inks (green, red, and black Sharpie ultra fine point marker) eventually delaminate from the tape at different rates when wetted, forming a local void at the interface between the patterned lines and the tape as the only path for liquid to flow. The capillary flow is delayed for a desired period until the void becomes large enough to allow the flow to proceed, acting as a timer. The void forming rates (i.e., delamination rates) depend on the material properties of delaminating ink, such as hydrophobicity and interfacial adhesion energy. The timer patterned with green delaminating ink expired the fastest, and the black timer was the slowest, producing varying delays: green <red <black timer. Different flow delays were assigned to each paper layer by imprinting different colored timers at strategic nodes, resulting in automated sequential flow following a programmed sequence.

### Fabrication of Device Case

The 3D design of the device case was created using Solidworks software (SolidWorks Corp., Waltham, MA) and printed using a 3D printer (FormLabs Form 3B) with High Temp V2 Resin as a structural material. The uncured resin was removed by soaking the printed device in isopropyl alcohol (IPA) for 15 min and the supporting structures were cut out. The device surface was then rendered smoothly with sandpaper and washed with DI water. After drying, white paint (Rust-Oleum, Painters Touch 2× Spray Paint Matte White) was sprayed on the device and fully dried.

### Saliva Sample Preparation

Saliva samples were collected from healthy donors according to a protocol approved by IRB of Georgia Institute of Technology. PCR confirmed COVID-19 patient saliva samples were purchased from Lee Biosolutions, Inc. (Maryland Heights, MO, USA). Recombinant nucleocapsid proteins (NP) of SARS-CoV-2 and influenza A and B viruses were obtained from MyBioSource (San Diego, CA, USA). Controlled amounts of each recombinant NP were spiked into the collected saliva. The spiked samples were serially diluted with saliva to set the desired concentrations when mixed with the virus extraction solution: 0.08 to 250 μg/ml for SARS-CoV-2 samples and 0.04 to 125 ng/ml for influenza A and B samples.

### Commercial LFAs and Signal Amplification Reagent

To enable signal amplification on LFA via autometallographic reaction, LFA should be a platform based on gold nanoparticles (AuNPs). Two commercial AuNP-based LFAs for SARS-CoV-2 (iHealth COVID-19 Antigen Rapid Test) and influenza A &B LFA (BD Veritor System Flu A+B) were purchased from iHealth Lab, Inc. (Mountain View, CA, USA) and BD Diagnostics (Sparks, MD, USA), respectively. Gold enhancement solution (Nanoprobes, Yaphank, NY) as the reagent A and B needed for signal amplification was obtained from Nanoprobes (Yaphank, NY, USA).

### Quantification of Color Intensity

The colorimetric signal intensities at the test and control lines were quantified using the ImageJ program.36 The raw images captured before and after the signal amplification were converted to monochrome, and gray values were measured at the test/control lines and in the background.22 Measured gray values were then used to calculate the normalized intensity based on the following equation Normalized intensity

$$Normalized intensity = \frac{{I}_{t} - {I}_{b}}{{I}_{c} - {I}_{b}}$$

where It, Ic, and Ib are the measured gray values at the test, control line, and background, respectively.

### REFERENCES

1. Giuntella, O.; Hyde, K.; Saccardo, S.; Sadoff, S. Lifestyle and Mental Health Disruptions during COVID-19. Proc. Natl. Acad. Sci. U.S.A 2021, 118, e2016632118.
2. Zhang, J.; Litvinova, M.; Liang, Y; Wang, Y; Wang, W.; Zhao, S.; Wu, Q.; Merler, S.; Viboud, C.; Vespignani, A.; et al. Changes in Contact Patterns Shape the Dynamics of the COVID-19 Outbreak in China. Science 2020, 368, 1481-1486.
3. Chinazzi, M.; Davis, J. T.; Ajelli, M.; Gioannini, C.; Litvinova, M.; Merler, S.; Pastore y Piontti, A.; Mu, K.; Rossi, L.; Sun, K.; et al. The Effect of Travel Restrictions on the Spread of the 2019 Novel Coronavirus (COVID-19) Outbreak. Science 2020, 368, 395-400.
4. Hampshire, A.; Hellyer, P. J.; Soreq, E.; Mehta, M. A.; Ioannidis, K.; Trender, W.; Grant, J. E.; Chamberlain, S. R. Associations between Dimensions of Behaviour, Personality Traits, and Mental-Health during the COVID-19 Pandemic in the United Kingdom. Nat. Commun. 2021, 12, 4111.
5. Hartley, S. L.; Fleming, V.; Piro-Gambetti, B.; Cohen, A.; Ances, B. M.; Yassa, M. A.; Brickman, A. M.; Handen, B. L.; Head, E.; Mapstone, M.; et al. Impact of the COVID-19 Pandemic on Daily Life, Mood, and Behavior of Adults with Down Syndrome. Disabil. Health J. 2022, 15, 101278.
6. Sigal, A.; Milo, R.; Jassat, W. Estimating Disease Severity of Omicron and Delta SARS-CoV-2 Infections. Nat. Rev. Immunol. 2022, 22, 267-269.
7. Andeweg, S. P.; de Gier, B.; Eggink, D.; van den Ende, C.; Maarseveen, N. Van; Ali, L.; Vlaemynck, B.; Schepers, R.; Hahne, S. J. M.; Reusken, C. B. E. M.; et al. Protection of COVID-19 Vaccination and Previous Infection against Omicron BA. 1, BA.2 and Delta SARS-CoV-2 Infections. Nat. Commun. 2022, 13, 4738.
8. Bowen, J. E.; Addetia, A.; Dang, H. V.; Stewart, C.; Brown, J. T.; Sharkey, W. K.; Sprouse, K. R.; Walls, A. C.; Mazzitelli, I. G.; Logue, J. K.; et al. Omicron Spike Function and Neutralizing Activity Elicited by a Comprehensive Panel of Vaccines. Science 2022, eabq0203.
9. Luo, S.; Zhang, J.; Kreutzberger, A. J. B.; Eaton, A.; Edwards, R. J.; Jing, C.; Dai, H.; Sempowski, G. D.; Cronin, K.; Parks, R.; et al. An Antibody from Single Human VH-Rearranging Mouse Neutralizes All SARS-CoV-2 Variants Through BA. 5 by Inhibiting Membrane Fusion. Sci. Immunol. 2022, eadd5446.
10. Wang, Q.; Guo, Y; Iketani, S.; Nair, M. S.; Li, Z.; Mohri, H.; Wang, M.; Yu, J.; Bowen, A. D.; Chan, J. Y; et al. Antibody Evasion by SARS-CoV-2 Omicron Subvariants BA.2.12.1, BA.4, and BA.5. Nature 2022.
11. Cao, Y; Yisimayi, A.; Jian, F.; Song, W.; Xiao, T.; Wang, L.; Du, S.; Wang, J.; Li, Q.; Chen, X.; et al. BA.2.12.1, BA.4 and BA.5 Escape Antibodies Elicited by Omicron Infection. Nature 2022.
12. Menni, C.; Valdes, A. M.; Freidin, M. B.; Sudre, C. H.; Nguyen, L. H.; Drew, D. A.; Ganesh, S.; Varsavsky, T.; Cardoso, M. J.; El-Sayed Moustafa, J. S.; et al. Real-Time Tracking of Self-Reported Symptoms to Predict Potential COVID-19. Nat. Med. 2020, 26, 1037-1040.
13. Carter, L. J.; Garner, L. V.; Smoot, J. W.; Li, Y; Zhou, Q.; Saveson, C. J.; Sasso, J. M.; Gregg, A. C.; Soares, D. J.; Beskid, T. R.; et al. Assay Techniques and Test Development for COVID-19 Diagnosis. ACS Cent. Sci. 2020, 591-605.
14. Udugama, B.; Kadhiresan, P.; Kozlowski, H. N.; Malekjahani, A.; Osborne, M.; Li, V. Y C.; Chen, H.; Mubareka, S.; Gubbay, J. B.; Chan, W. C. W. Diagnosing COVID-19: The Disease and Tools for Detection. ACS Nano 2020, 14, 3822-3835.
15. Fan, Y; Li, X.; Zhang, L.; Wan, S.; Zhang, L.; Zhou, F. SARS-CoV-2 Omicron Variant: Recent Progress and Future Perspectives. Signal Transduct. Target. Ther. 2022, 7, 141.
16. Sah, P.; Fitzpatrick, M. C.; Zimmer, C. F.; Abdollahi, E.; Juden-Kelly, L.; Moghadas, S. M.; Singer, B. H.; Galvani, A. P. Asymptomatic SARS-CoV-2 Infection: A Systematic Review and Meta-Analysis. Proc. Natl. Acad. Sci. U.S.A 2021, 118, e2109229118.
17. Shang, W.; Kang, L.; Cao, G.; Wang, Y; Gao, P.; Liu, J.; Liu, M. Percentage of Asymptomatic Infections among SARS-CoV-2 Omicron Variant-Positive Individuals: A Systematic Review and Meta-Analysis. Vaccines 2022, 10, 1049.
18. Johansson, M. A.; Quandelacy, T. M.; Kada, S.; Prasad, P. V; Steele, M.; Brooks, J. T.; Slayton, R. B.; Biggerstaff, M.; Butler, J. C. SARS-CoV-2 Transmission from People without COVID-19 Symptoms. JAMANetw. Open 2021, 4, e2035057.
19. Subramanian, R.; He, Q.; Pascual, M. Quantifying Asymptomatic Infection and Transmission of COVID-19 in New York City Using Observed Cases, Serology, and Testing Capacity. Proc. Natl. Acad. Sci. U.S.A 2021, 118, e2019716118.
20. Gong, M. M.; Sinton, D. Turning the Page: Advancing Paper-Based Microfluidics for Broad Diagnostic Application. Chem. Rev. 2017, 117, 8447-8480.
21. Yetisen, A. K.; Akram, M. S.; Lowe, C. R. Paper-Based Microfluidic Point-of-Care Diagnostic Devices. Lab Chip 2013, 13, 2210-2251.
22. Lee, D.; Ozkaya-Ahmadov, T.; Chu, C. H.; Boya, M.; Liu, R.; Sarioglu, A. F. Capillary Flow Control in Lateral Flow Assays via Delaminating Timers. Sci. Adv. 2021, 7, eabf9833.
23. Zhou, Y; Wu, Y; Ding, L.; Huang, X.; Xiong, Y Point-of-Care COVID-19 Diagnostics Powered by Lateral Flow Assay. Trends Anal. Chem. 2021, 145, 116452.
24. Zhang, Y; Chai, Y; Hu, Z.; Xu, Z.; Li, M.; Chen, X.; Yang, C.; Liu, J. Recent Progress on Rapid Lateral Flow Assay-Based Early Diagnosis of COVID-19. Front. Bioeng. Biotechnol. 2022, 10, 866368.
25. Hsiao, W. W. W.; Le, T. N.; Pham, D. M.; Ko, H. H.; Chang, H. C.; Lee, C. C.; Sharma, N.; Lee, C. K.; Chiang, W. H. Recent Advances in Novel Lateral Flow Technologies for Detection of COVID-19. Biosensors 2021, 11, 295.
26. Amanat, F.; Stadlbauer, D.; Strohmeier, S.; Nguyen, T. H. O.; Chromikova, V.; McMahon, M.; Jiang, K.; Arunkumar, G. A.; Jurczyszak, D.; Polanco, J.; et al. A Serological Assay to Detect SARS-CoV-2 Seroconversion in Humans. Nat. Med. 2020, 26, 1033-1036.
27. Krammer, F.; Simon, V. Serology Assays to Manage COVID-19. Science 2020, 368, 1060-1061.
28. Trombetta, B. A.; Kandigian, S. E.; Kitchen, R. R.; Grauwet, K.; Webb, P. K.; Miller, G. A.; Jennings, C. G.; Jain, S.; Miller, S.; Kuo, Y; et al. Evaluation of Serological Lateral Flow Assays for Severe Acute Respiratory Syndrome Coronavirus-2. BMC Infect. Dis. 2021, 21, 580.
29. de Lima, L. F.; Ferreira, A. L.; Torres, M. D. T.; de Araujo, W. R.; de la Fuente-Nunez, C. Minute-Scale Detection of SARS-CoV-2 Using a Low-Cost Biosensor Composed of Pencil Graphite Electrodes. Proc. Natl. Acad. Sci. U.S.A 2021, 118, e2106724118.
30. Service, R. F. Coronavirus Antigen Tests: Quick and Cheap, but Too Often Wrong?Science 2020.
31. Bai, Z.; Cao, Y; Liu, W.; Li, J. The SARS-CoV-2 Nucleocapsid Protein and Its Role in Viral Structure, Biological Functions, and a Potential Target for Drug or Vaccine Mitigation. Viruses 2021, 13, 1115.
32. Thura, M.; En Sng, J. X.; Ang, K. H.; Li, J.; Gupta, A.; Hong, J. M.; Hong, C. W.; Zeng, Q. Targeting Intra-viral Conserved Nucleocapsid (N) Proteins as Novel Vaccines against SARS-CoVs. Biosci. Rep. 2021, 41, BSR20211491.
33. Dutta, N. K.; Mazumdar, K.; Gordy, J. T. The Nucleocapsid Protein of SARS-CoV-2: A Target for Vaccine Development. J. Virol. 2020, 94, e00647-20.
34. Shan, D.; Johnson, J. M.; Fernandes, S. C.; Suib, H.; Hwang, S.; Wuelfing, D.; Mendes, M.; Holdridge, M.; Burke, E. M.; Beauregard, K.; et al. N-Protein Presents Early in Blood, Dried Blood and Saliva during Asymptomatic and Symptomatic SARS-CoV-2 Infection. Nat. Commun. 2021, 12, 1931.
35. Lee, D.; Chu, C. H.; Sarioglu, A. F. Point-of-Care Toolkit for Multiplex Molecular Diagnosis of SARS-CoV-2 and Influenza A and B Viruses. ACS Sens. 2021, 6, 3204-3213.
36. Schneider, C. A.; Rasband, W. S.; Eliceiri, K. W. NIH Image to ImageJ: 25 years of image. Nat Methods, 2012, 9, 671-675.

### Example 3. Fluidic Diode Through Asymmetric Design of Delaminating Timer

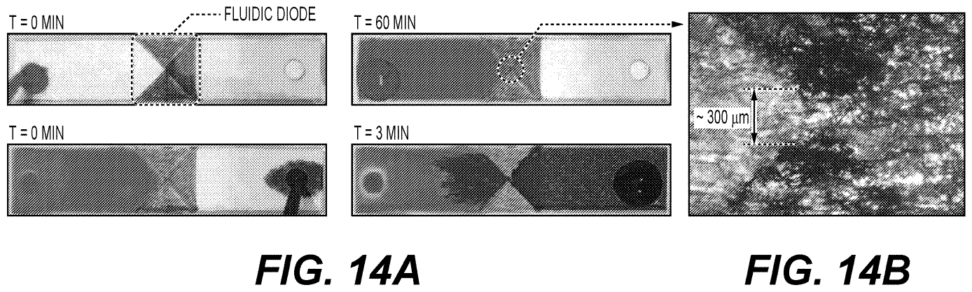

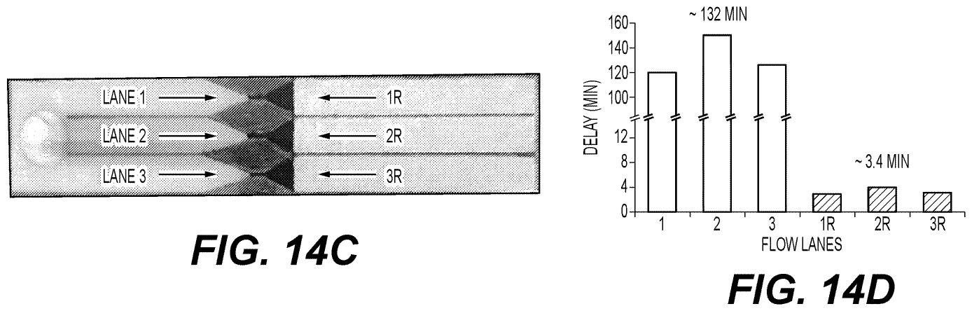

The asymmetric design of a delaminating timer can be used to create a fluidic diode. By reducing the contact area of the fluid facing the timer, the exemplary system and method can drastically increase the flow delay and it further increases as the contact area decreases more. As an example of asymmetric design, the study created a triangular timer with green delaminating ink (FIG. 14A). The left fluidic channel was facing the vertex of the triangular timer (300 μm width, FIG. 14B), and the right channel was directly connected to the wide side (4 mm) of the timer. The study observed that the yellow dye solution introduced from the left inlet could not flow through the triangular timer and the flow stopped at the entrance of the timer for >60 min. On the other hand, within the same device, the red dye solution applied to the right inlet could flow through the triangular timer in 3 min. For quantitative measurement of the nonlinear delay in the triangular timer, the study created a test layout where three fluidic lanes were equipped with identical triangular timers drawn with black delaminating ink which creates the longest flow delay (FIG. 14C). While the capillary flow starting from the left in the three lanes took 132 min to pass through the timer, the flow introduced from the right only took~3.4 min (FIG. 14D), showing ~39 times difference in the flow delay. The significant difference in delay depending on the introducing direction of flows can be used to create a fluidic diode.

### Computer Simulation of Fluidic Diode

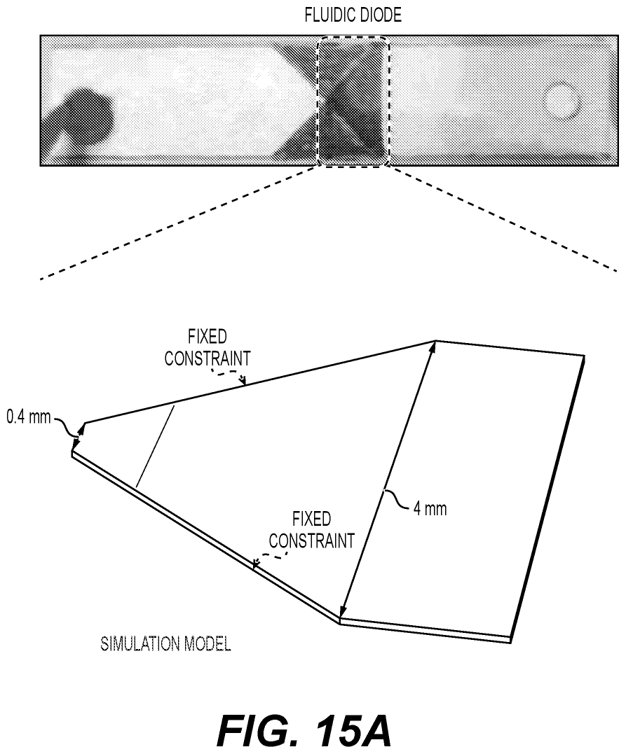

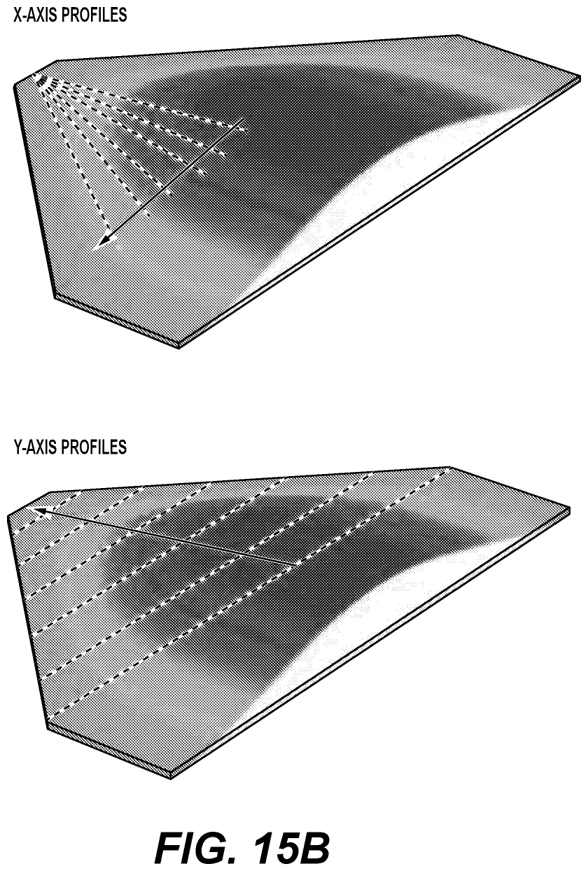

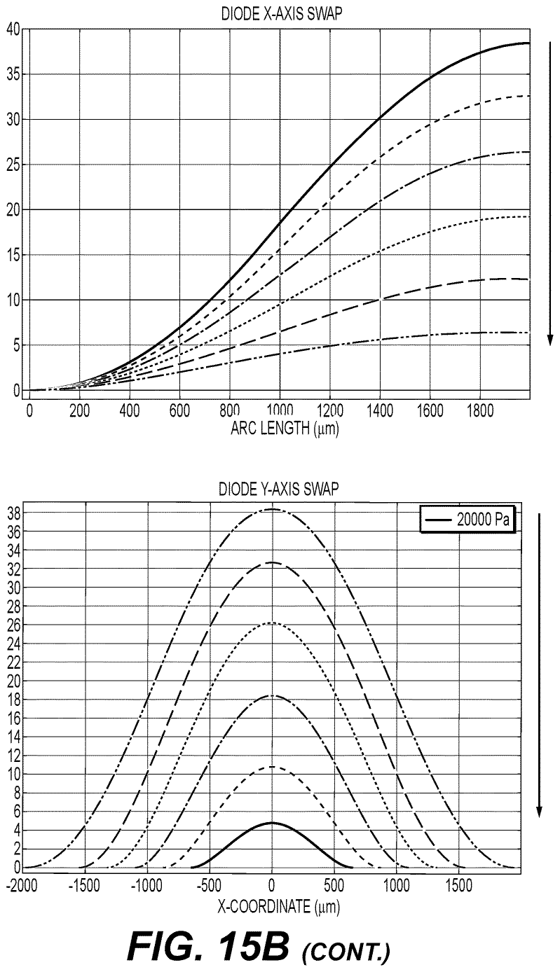

To better understand the principle of the fluidic diode, the study conducted the computer simulation with the asymmetric design of the timer (FIG. 15) using COMSOL Multiphysics v5.6 (COMSOL, MA, USA). Solid mechanics was used for the main physics with 40 μm of acrylic plastic as backing material of the tape (density: 1190 kg/m3, Young's modulus: 2.7e9 Pa, Poisson's ratio: 0.35). In the asymmetric design, the study built the trapezoid shape of timer (short base: 0.4 mm, long base: 4 mm, height: 2 mm) and fixed constraints were applied to all of side edges. Then, uniform pressure were applied across the built model and measured the change of the surface profile in the x and y-axis. The study confirmed that the surface profile formed a dome shape, and the surface height was gradually decreased from long base to short base as well as from center to edge. From this result, it was observed if the base length in the timer becomes narrower, it becomes harder to delaminate under the same pressure.

### Fluidic Transistor Using Back-to-Back Diodes

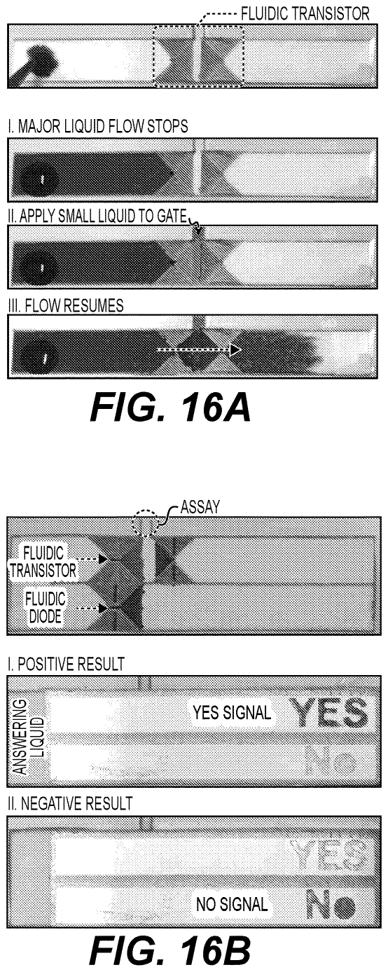

By placing two fluidic diodes in a back-to-back manner, the exemplary system and method can create a fluidic transistor, the major (large) liquid flow can be controlled by a minor (small) liquid application. In the prototype of a device (FIG. 16A), the large flow of red dye solution applied to the main fluidic channel stopped due to the fluidic transistor (FIG. 16A, i). The introduction of the small controlling liquid (~3 μL) to the gate (FIG. 16A, ii) delaminated the back-to-back diodes and made the major liquid flow resume by passing through the transistor design (FIG. 16A, iii). The concept of the fluidic transistor can be applied to analytical assay applications by controlling the release time of small liquid or using a certain assay that can produce a small amount of liquid as a byproduct. As an example of device configuration, the study designed a ‘Yes-or-No-answering’ type of assay (FIG. 16B). The presence of a target analyte in the sample can release or produce a small amount of liquid at the gate, which results in the release of an answering liquid to ‘YES’ channel equipped with the fluidic transistor, representing a positive result (FIG. 16B, i). The negative result with no target analyte cannot release the answering liquid to the ‘YES’ channel within the assay time but release it to the ‘No’ channel eventually (FIG. 16B, ii).

### Clinical Application of Fluidic Diode

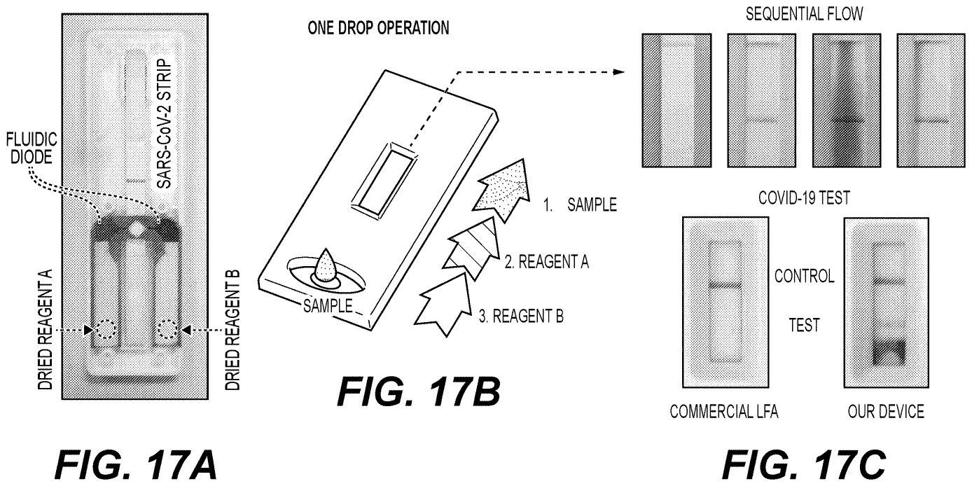

As one of the clinical applications of the fluidic diode, the study created a lateral flow assay (LFA) with built-in signal amplification that can both run an assay and amplify the result by simply dropping the sample. Rather than applying the sample and signal amplification reagents manually to separate inlets, one dropping action of the sample can perform both the sample transfer to the LFA strip for assay and sequential delivery of the amplification reagents through rehydration of reagents as the sample is used for a liquid carrier. The device was built by incorporating a commercial LFA strip for SAR_S-CoV-2 assay with the paper-based flow controller equipped with green and black fluidic diode (FIG. 17A): black diode generates a longer flow delay. When only applying the sample into the inlet by one dropping action (FIG. 17B), the sample initially flowed to the LFA strip, and the two fluidic diodes prevent the backflow of the sample already introduced to the LFA strip to the flow controller. Meanwhile, the sample applied to two side channels of the flow controller rehydrated the dried amplification reagents A and B, and the reagents were sequentially delivered to the LFA strip in the designed order to amplify the colorimetric signal. Compared to the commercial LFA for the COVID-19 test, the device could achieve enhanced sensitivity even with the use of the same concentration of the sample, reducing the risk of false negatives (FIG. 17C).

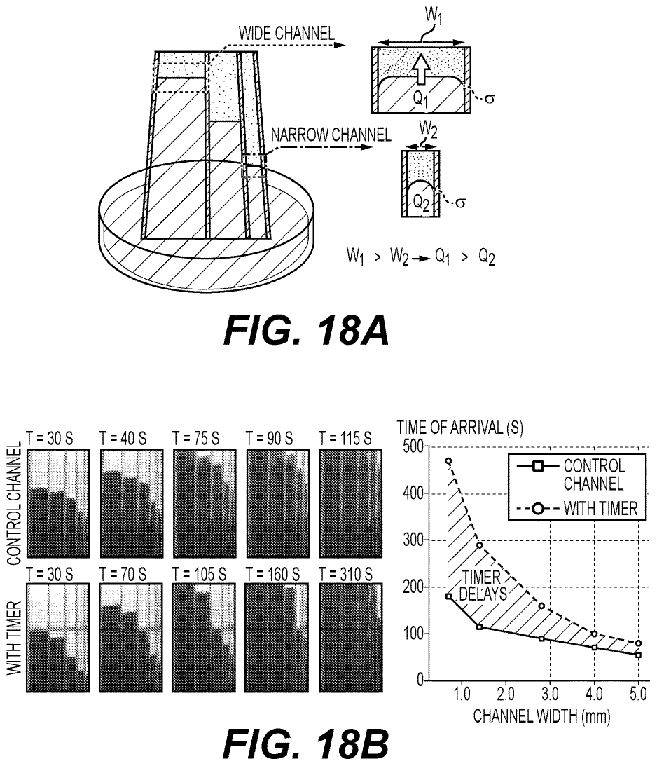

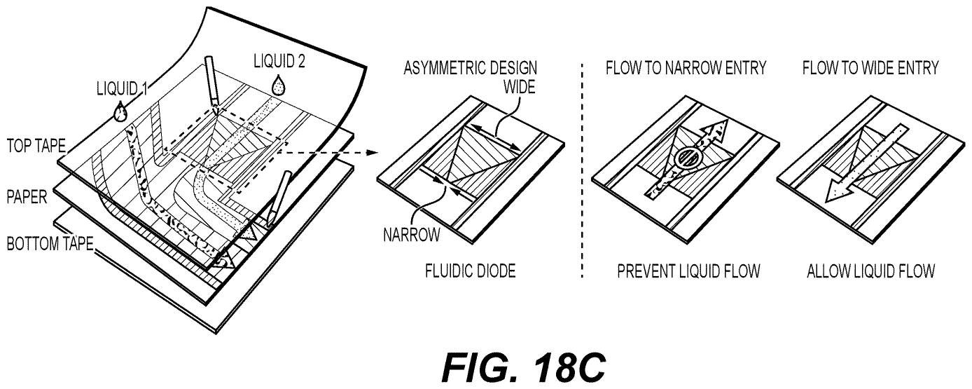

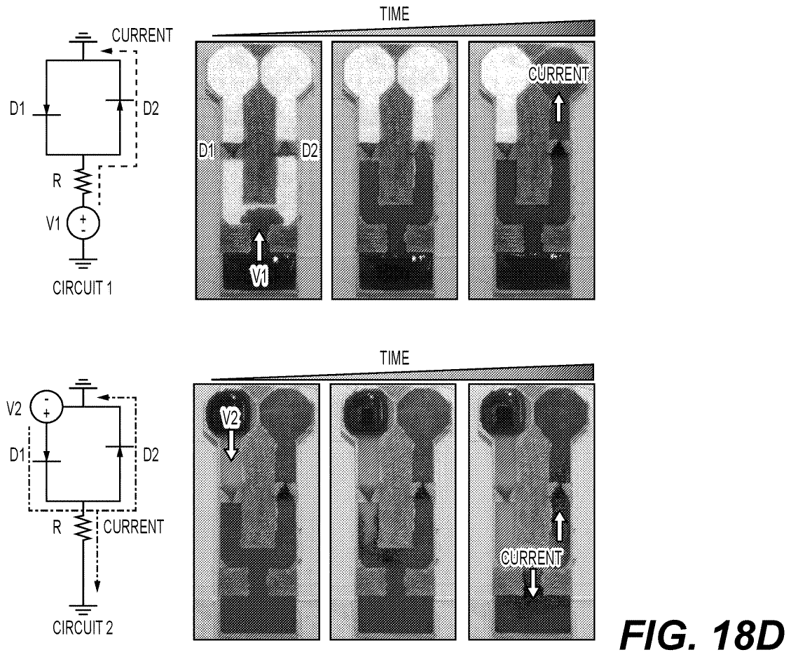

FIG. 18A-18D illustrates the concept and underlying principle of the fluidic diode. When a channel is patterned on paper using hydrophobic material (FIG. 18A, brown lines), the flow rate (Q) within the channel becomes contingent on the channel width. This dependency arises from the fact that hydrophobic boundaries, created by the hydrophobic material, substantially diminish the driving forces facilitated by capillarity. The surface tension (σ) at these boundaries acts in an opposing direction to the flow, thereby retarding the imbibition speed. Consequently, the flow rate (Q₂) in the narrower channel (w₂) is lower than that (Q₁) in the wider channel (w₁) (FIG. 18A). On top of this basics, a delaminating timer was introduced to the paper channels of varying widths and examined the impact of channel width on the timer's delays (FIG. 18B). In control channels, which featured a gradual reduction in width across 5 channels, the narrower channels showed a lower flow rate as anticipated (black line in graph). In channels outfitted with the same thickness as the timer, there was observed an exponential increase in the timer's delays as the channel width decreased (red line in graph). Expanding on these fundamental principles, the study devised an asymmetric design of the delaminating timer (FIG. 18C), called the fluidic diode, which functions analogously to an electrical diode. In this configuration, a liquid introduced into the wider entry effortlessly traverses the fluidic diode, akin to the forward biasing in an electrical diode. Conversely, when a liquid is introduced into the narrower entry, it experiences a significantly prolonged passage through the fluidic diode, mirroring the behavior of reverse biasing in an electrical diode. To experimentally demonstrate the function of the fluidic diode, the study devised a test platform featuring two fluidic diodes oriented in opposite directions (FIG. 18D). In both designs (circuits 1 and 2), liquids could only pass through the fluidic diode facing the wider entry: red liquid could only pass through a diode 2 (D2), while blue liquid could only pass a diode 1 (D1).

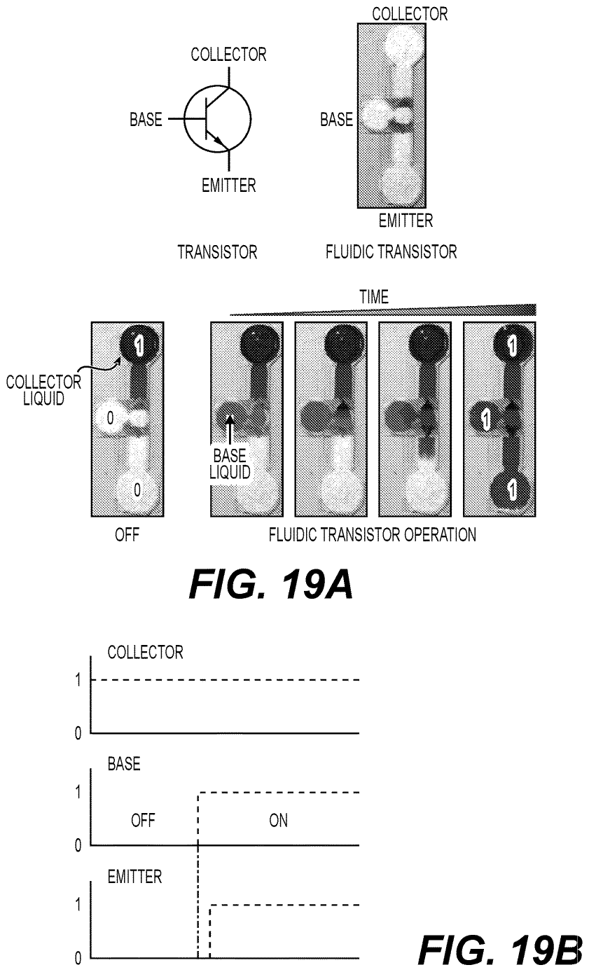

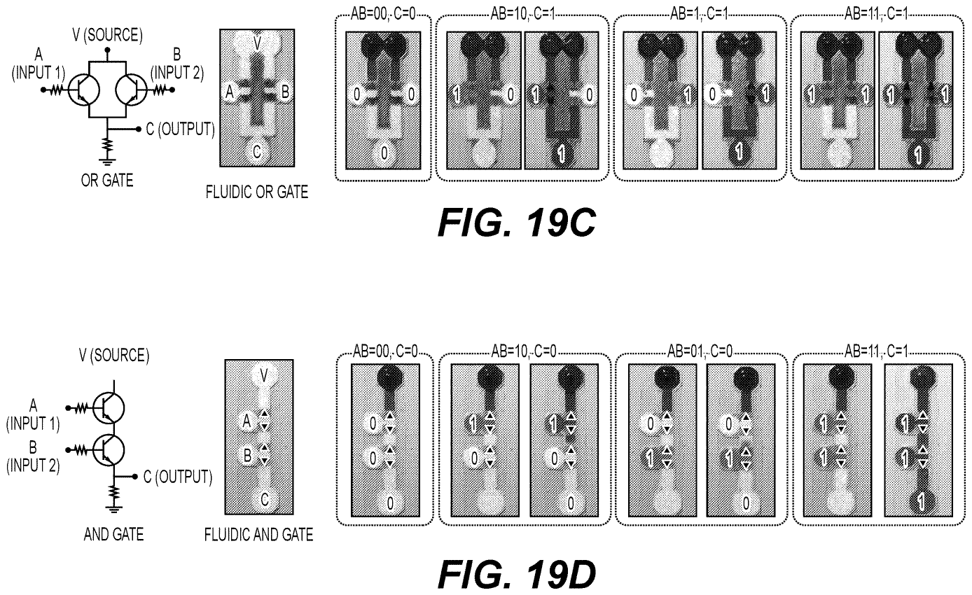

With the strategic arrangement of multiple fluidic diodes, it becomes feasible to implement fluidic transistors and logic gates on paper that behave similarly to electrical transistors (FIG. 19A) and logic gates (FIGS. 19C and 19D). In the configuration of the fluidic transistor, two fluidic diodes were positioned in opposite directions within the same channel, creating a base channel between them. The introduction of liquid (red) to the collector did not permit its flow to the emitter side unless liquid (yellow) was introduced to the base. Activation of the two fluidic diodes by the liquid (yellow) in the base allowed the liquid (red) to flow towards the emitter.

In the configuration of the fluidic OR gate, two fluidic transistor were positioned in parallel, sharing a common source and output. Similar to the behavior of an electrical OR gate, the introduction of a liquid (yellow) in at least one of the inputs, A (input 1) or B (input 2), produced the output state of 1 (red liquid) and only if both inputs are 0 (no liquid), the output will represent 0 (no liquid). Similarly, in the configuration of the fluidic AND gate, two fluidic transistor were connected in serial. As similar behavior of an electrical AND gate, the introduction of a liquid (yellow) in both the inputs, A (input 1) or B (input 2), produced the output state of 1 (red liquid) and if either A (input 1) or B (input 2) is 0 (no liquid), the output will represent 0 (no liquid).

1. A method to manipulate capillary-driven flow, the method comprising: receiving a liquid sample at a sample inlet of a microfluidic device;
flowing the liquid sample along a capillary channel disposed in a substrate of the microfluidic device to a flow delay section of the substrate defined by an area, the area having a delaminating ink forming water-penetrable bonds infused therein;
halting the flow of the liquid sample along the substrate at the flow delay section;
delaminating the delaminating ink in the area, via a liquid sample interaction with the delaminating ink at a controlled rate to produce flow delay of the liquid sample at the flow delay section; and
flowing the liquid sample along another section of the substrate following the flow delay section after a controlled pre-defined time,
wherein the liquid sample is subsequently mixed with at least one reagent located in the microfluidic device for analysis (e.g., colorimetry analysis, assay test, chemical amplification) or visualization (e.g., art), and wherein the flow delay section provides a pre-defined time delay for the analysis;
wherein the flow delay section comprises a diode defined by a taper shaped delaminating ink disposed between two taper shaped non-delaminating ink, wherein the two taper shaped non-delaminating ink are perpendicular to each other, wherein the delaminating ink has a first contact region that expands to a second contact region larger than the first contact region.

2. The method of claim 1, wherein the device further comprises a plurality of ink-based transistors arranged in a serial arrangement or parallel arrangement.

3. The method of claim 2, wherein the ink-based transistor is formed by having at least two diodes opposite to each other, wherein flow is controlled by liquid application at a gate between the two fluidic diodes.

4. The method of claim 3, wherein the ink-based transistor comprises an input, a gate, and an output, wherein a fluid flow to the gate allows the liquid sample to flow from the input to the output.

5. The method of claim 1, wherein the reagent amplifies a colorimetric signal.

6. The method of claim 1, wherein the liquid sample is subsequently mixed with at least one reagent located in the microfluidic device for visualization.

7. The method of claim 1, further comprising:
guiding the flow of the liquid sample along the capillary channel and/or flow delay section with a non-delaminating ink infused into the substrate to form boundaries of the capillary channel and/or flow delay section.

8. The method of claim 1, wherein the capillary channel comprises a first line of a non-delaminating ink and a second line of the non-delaminating ink infused on the substate.

9. The method of claim 8, wherein flow delay section comprises a line of delaminating ink extending between the first line and second line of non-delaminating ink.

10. The method of claim 9, wherein the flow delay section comprises more than two lines of the delaminating ink extending between the first line and the second line of non-delaminating ink.

11. The method of claim 1, wherein the non-delaminating ink comprises a hydrophobic resin.

12. The method of claim 1, wherein the microfluidic device further comprises laminating tape adhering to the substrate to define a top and bottom boundaries for capillary flow.

13. The method of claim 1, further comprising:
flowing the liquid sample along a second capillary channel; and
testing the liquid sample of second capillary channel in the analysis or visualization prior to the liquid sample of the first capillary channel due to the pre-defined time delay provided to the first capillary channel by the flow delay section.

14. The method of claim 1, wherein a contact angle between the delaminating ink and the substrate is from 0° to 180°.

15. The method of claim 1, wherein the substrate is treated with a biomolecule.

16. The method of claim 1, wherein the device comprises a lateral flow assay.

17. The method of claim 1, wherein the sample comprises urine, blood, plasma, serum, sweat, saliva, or any combination thereof.

18. A microfluidic device comprising:
a substrate configured to move a fluid by capillary action, the substrate having an inlet area, a test area, and a flow area;
adhesive polymetric material adhering to the substrate to define a top and bottom boundaries for capillary flow;
a capillary channel disposed in the substrate, the capillary channel defined by a first line of a non-delaminating ink and a second line of the non-delaminating ink infused on the substrate; and
a flow delay section defined by an area of the capillary channel, the area having a delaminating ink,
wherein the flow delay section comprises a diode defined by a taper shaped delaminating ink disposed between two taper shaped non-delaminating ink, wherein the two taper shaped non-delaminating ink are perpendicular to each other,
wherein the delaminating ink has a first contact region that expands to a second contact region larger than the first contact region, the delaminating ink being configured to delaminate, via fluid interaction with the delaminating ink, at a controlled rate to produce flow delay of the fluid at the flow delay section.

19-38. (canceled)

39. A method of detecting an infectious agent, comprising
applying a sample on the device of claim 18;
detecting a signal on the device.

40. The method of claim 39, wherein the infectious agent is a virus.

41-44. (canceled)


SEQUENCE LISTING (24 sequences)

SEQ ID NO: 1 - DNA, 21 bp, synthetic construct
  misc_feature 1..21: note = synthetic construct
  source 1..21: mol_type = other DNA
accaggaactaatcagacaag

SEQ ID NO: 2 - DNA, 25 bp, synthetic construct
  misc_feature 1..25: note = synthetic construct
  source 1..25: mol_type = other DNA
gacttgatctttgaaatttggatct

SEQ ID NO: 3 - DNA, 42 bp, synthetic construct
  misc_feature 1..42: note = synthetic construct
  source 1..42: mol_type = other DNA
ttccgaagaacgctgaagcggaactgattacaaacattggcc

SEQ ID NO: 4 - DNA, 40 bp, synthetic construct
  misc_feature 1..40: note = synthetic construct
  source 1..40: mol_type = other DNA
cgcattggcatggaagtcacaatttgatggcacctgtgta

SEQ ID NO: 5 - DNA, 21 bp, synthetic construct
  misc_feature 1..21: note = synthetic construct
  source 1..21: mol_type = other DNA
gggggcaaattgtgcaatttg

SEQ ID NO: 6 - DNA, 20 bp, synthetic construct
  misc_feature 1..20: note = synthetic construct
  source 1..20: mol_type = other DNA
cttcgggaacgtggttgacc

SEQ ID NO: 7 - DNA, 23 bp, synthetic construct
  misc_feature 1..23: note = synthetic construct
  source 1..23: mol_type = other DNA
tgagtacgaacttatgtactcat

SEQ ID NO: 8 - DNA, 22 bp, synthetic construct
  misc_feature 1..22: note = synthetic construct
  source 1..22: mol_type = other DNA
ttcagatttttaacacgagagt

SEQ ID NO: 9 - DNA, 42 bp, synthetic construct
  misc_feature 1..42: note = synthetic construct
  source 1..42: mol_type = other DNA
accacgaaagcaagaaaaagaagttcgtttcggaagagacag

SEQ ID NO: 10 - DNA, 44 bp, synthetic construct
  misc_feature 1..44: note = synthetic construct
  source 1..44: mol_type = other DNA
ttgctagttacactagccatccttaggttttacaagactcacgt

SEQ ID NO: 11 - DNA, 18 bp, synthetic construct
  misc_feature 1..18: note = synthetic construct
  source 1..18: mol_type = other DNA
cgctattaactattaacg

SEQ ID NO: 12 - DNA, 19 bp, synthetic construct
  misc_feature 1..19: note = synthetic construct
  source 1..19: mol_type = other DNA
gcgcttcgattgtgtgcgt

SEQ ID NO: 13 - DNA, 19 bp, synthetic construct
  misc_feature 1..19: note = synthetic construct
  source 1..19: mol_type = other DNA
gctaagagagcaattgagc

SEQ ID NO: 14 - DNA, 19 bp, synthetic construct
  misc_feature 1..19: note = synthetic construct
  source 1..19: mol_type = other DNA
atgtaggatttgctgagct

SEQ ID NO: 15 - DNA, 44 bp, synthetic construct
  misc_feature 1..44: note = synthetic construct
  source 1..44: mol_type = other DNA
cgagtcatgattgggccatgacagtgtcatcatttgaaaggttt

SEQ ID NO: 16 - DNA, 46 bp, synthetic construct
  misc_feature 1..46: note = synthetic construct
  source 1..46: mol_type = other DNA
aaggtgtaacggcagcatgtccgaatttccttttttaactagccat

SEQ ID NO: 17 - DNA, 21 bp, synthetic construct
  misc_feature 1..21: note = synthetic construct
  source 1..21: mol_type = other DNA
acttgtcttggggaatatctc

SEQ ID NO: 18 - DNA, 18 bp, synthetic construct
  misc_feature 1..18: note = synthetic construct
  source 1..18: mol_type = other DNA
atgctggagcaaaaagct

SEQ ID NO: 19 - DNA, 20 bp, synthetic construct
  misc_feature 1..20: note = synthetic construct
  source 1..20: mol_type = other DNA
agggacatgaacaacaaaga

SEQ ID NO: 20 - DNA, 19 bp, synthetic construct
  misc_feature 1..19: note = synthetic construct
  source 1..19: mol_type = other DNA
caagtttagcaacaagcct

SEQ ID NO: 21 - DNA, 49 bp, synthetic construct
  misc_feature 1..49: note = synthetic construct
  source 1..49: mol_type = other DNA
tcagggacaatacattacgcatatcgataaaggaggaagtaaacactca

SEQ ID NO: 22 - DNA, 43 bp, synthetic construct
  misc_feature 1..43: note = synthetic construct
  source 1..43: mol_type = other DNA
taaacggaacattcctcaaacaccactctggtcataggcattc

SEQ ID NO: 23 - DNA, 23 bp, synthetic construct
  misc_feature 1..23: note = synthetic construct
  source 1..23: mol_type = other DNA
tcaaacggaacttcccttctttc

SEQ ID NO: 24 - DNA, 25 bp, synthetic construct
  misc_feature 1..25: note = synthetic construct
  source 1..25: mol_type = other DNA
ggatacaagtccttatcaactctgc